(19) United States
(12) Patent Application Publication
(10) Pub. No.: US 2025/0263646 A1
(43) Pub. Date: Aug. 21, 2025

(54) TUMOR MICROENVIRONMENT MIMICKING DEVICE AND METHOD

(71) Applicant: UNIVERISTY OF CYPRUS, Aglantizia Nicosia (CY)

(72) Inventors: Paris A. Skourides, Aglantizia Nicosia (CY); Adonis Hadjigeorgiou, Aglantizia Nicosia (CY); Neophytos Christodoulou, Aglantizia Nicosia (CY)

(21) Appl. No.: 18/702,201

(22) PCT Filed: Nov. 14, 2022
(86) PCT No.: PCT/IB2022/060937

§ 371 Date: Apr. 17, 2024

Related U.S. Application Data
(60) Provisional application No. 63/279,314, filed on Nov. 15, 2021.

(51) Int. Cl. C12M 1/22; C12M 1/00; C12M 1/12; C12M 1/34; C12N 5/09

(52) U.S. Cl. CPC C12M 23/10; C12M 23/34; C12M 23/38; C12M 37/04; C12M 41/34; C12N 5/0693; C12N 2513/00

(57) ABSTRACT

The present disclosure provides tumor microenvironment mimicking (“TMM”) devices and in vitro methods that can accurately mimic a tumor microenvironment and be used to investigate tumor progression, especially with respect to tumor cell proliferation, metabolic activity, 3D migration and invasion, in vitro isolation of cancer cell subpopulations, and the gene expression changes leading to increased tumor aggressiveness. The TMM devices may comprise a base including a bottom wall and a plurality of base posts extending from the bottom wall, the base posts separated from each other to form spaces; and a lid sized to mate with the base and including a mating wall and a plurality of lid posts extending from the mating wall, the lid posts positioned so as to be able to variably cover the spaces when the lid is mated with the base depending on a rotational position of the lid relative to the base.

### CROSS REFERENCE TO RELATED APPLICATIONS

This application claims the benefit of U.S. Provisional Application 63/279,314 filed on Nov. 15, 2021, of which is hereby incorporated by reference in its entirety.

### TECHNICAL FIELD

The present disclosure relates generally to devices and in vitro methods for investigating tumor progression, especially with respect to tumor cell proliferation, metabolic activity, 3D migration and invasion, in vitro isolation of cancer cell subpopulations, and gene expression changes leading to increased tumor aggressiveness.

### BACKGROUND

The present disclosure generally relates to tumor microenvironment mimicking (“TMM”) devices, specifically, TMM devices and in vitro methods for investigating tumor progression, especially with respect to tumor cell proliferation, metabolic activity, 3D migration and invasion, and gene expression changes leading to increased tumor aggressiveness. In addition, the present disclosure relates to devices and in vitro methods for investigating metastatic potential, cellular interactions, cellular responses to drugs or compounds, and in particular, cellular responses in a setting that recapitulates the tumor 3D microenvironment of a solid tumor in vitro. Furthermore, the present disclosure relates to TMM devices and in vitro methods that allow the generation, isolation, and characterization of tumor cell subpopulations with more aggressive characteristics including higher metastatic potential, gene expression changes, epigenetic modifications and metabolic changes responsible for tumor progression.

The stepwise progression of human cancers is well documented and is clinically recognized. Pre-malignant lesions, such as dysplasia and hyperplasia, are detected in diverse organs prior to the appearance of fully malignant invasive tumors. The pre-malignant lesions are caused either by genetic alterations, which induce monoclonal expansion of the cells, or by environmental factors, such as viral infections. Thereafter, genetic alterations accumulate in one (or a few) of the pre-malignant cells, and these cells transform into malignant ones of clonal origin, giving rise to the primary tumor. At the early stages, tumor cells proliferate, and the primary tumor expands. However, at this stage, cancer cells are neither invasive nor metastatic. Subsequently, through the accumulation of genetic alterations, new clones develop within the cancer cell population, leading to acquisition of traits permissive for invasion and metastasis. Thus, cells in a primary tumor are phenotypically and biologically heterogeneous, with only a subset of cells in a primary tumor acquiring changes at the genomic level, which lead to distinct transcriptome signature, allowing them to be invasive and metastatic and this is rooted in differences in gene expression. Highly metastatic cells typically harbor a higher frequency of genomic mutations compared to non-metastatic cells leading to differential gene expression between these two cell populations. These cells selectively produce metastatic tumors in distant organs and are the primary cause of death in the vast majority of cancer patients. Thus, understanding and mapping genomic and transcriptomic alterations in cancer cells is of crucial importance since it will not only enhance our understanding of cancer cell progression, but will also reveal potential treatment targets, enabling early diagnosis and facilitating efficient treatment.

The gold standard to assess the metastatic ability of tumor cells involves the use of in vivo assays such as a mouse or rat model systems. In vivo assays are more time consuming and expensive than in vitro methods. While in vivo models are able to capture the complexity of the metastatic process in a living system, the study and visualization of the individual steps involved is challenging and requires infrastructure not accessible to the majority of research groups. In addition, extracting quantitative mechanistic data is typically very difficult and often not possible. Precisely tracking gene alterations and gene expression changes during tumor progression in animal models is further complicated by the fact that tumors are heterogeneous cell populations and contain various other cells including normal stromal and inflammatory cells. The presence of these cells often masks genetic and gene expression alterations. Similarly, isolation of metastatic tumors and examination of gene expression changes responsible for the acquisition of metastatic capacity is also complicated by gene expression changes which allow these cells to grow at a new distant site or a different organ, changes that do not necessarily reflect the initial acquisition of metastatic capacity. In contrast, in vitro models allow control of most experimental variables and permit quantitative analysis.

Existing in vitro methods and assays, however, are characterized by reduced physiological relevance, capturing only limited aspects of the tumor microenvironment. A spheroid assay is one example of an in vitro assay. Although the spheroid assay is the current standard for in vitro assays, spheroid assays have several limitations. For example, spheroid assays are not compatible with all cell lines, they do not have standardized protocols, and they have a limited capacity to mimic the tumor microenvironment.

For each of the above reasons, devices and methods that can better mimic the tumor microenvironment in vitro are needed.

### SUMMARY

The present disclosure includes reliable, simple, and low-cost 3D cell culture devices that are easy to use (having a straightforward protocol), fast, and provide highly reproducible results. The designs of the TMM devices in the present disclosure and its setup methodology allow the replication of the in vivo tumor microenvironment in vitro. The present TMM device has also uniquely been validated to allow tumor progression to take place and to be monitored outside of an animal, a critical feature never demonstrated before for any other in vitro assay or device. The TMM devices and in vitro methods of the present disclosure can be used to determine whether cells of interest are capable of migration and/or invasion within a relevant 3D environment, which is characterized by the presence of oxygen, pH, and nutrients gradients that are similar to the in vivo conditions. The TMM devices through intake restriction and device geometry lead to the de novo establishment of uniform stable radial gradients and to the generation of solid stress, through tumor cell confinement. In addition, the TMM devices and in vitro methods of the present disclosure can be used to analyze the effects that environmental factors and cells, which are typically found in the tumor microenvironment, such as cancer associated fibroblasts (CAFs) and tumor associated macrophages (TAMs), have on cancer cell migration and/or invasion. Further, the TMM devices and in vitro methods of the present disclosure may be used to study cancer progression and its cures. For example, the TMM devices and in vitro methods may be used to test anti-cancer drugs or other test compounds on metastatic cancer cells. The TMM devices and in vitro methods can also be used to study several aspects of cancer, such as cell to cell interactions, metastasis, extravasation, mechanical stimuli, ECM stiffness, the effects of cancer-associated fibroblast, metabolic activity of cancer cells, and the effects of different extracellular matrix components. Additionally, the TMM device and in vitro methods can be used for the isolation of heterogeneous cancer cell populations with higher metastatic potential compared to their parental cell line, which can be further analyzed to provide novel information and enhance our understanding of the molecular mechanism of metastasis that can lead to new treatment targets, enable early diagnosis, and facilitate treatment design. The TMM devices and in vitro methods of the present disclosure replicate the tumor microenvironment with such accuracy that cell populations introduced to the TMM device and exposed to its environmental stressors, uniquely display experimentally verified increased aggressiveness in preclinical, in vivo studies in mice, and increased expression of known oncogenes and down-regulation of tumor-suppressor genes when compared to the parental cell line.

Accurate representation of an in vivo tumor microenvironment of the present disclosure provides a powerful tool for studying cancer cells and evaluating potential therapeutics in a physiologically relevant 3D environment. The ability to visualize such processes via live microscopy and/or use other techniques to label, identify, or measure the cellular responses or effects provides numerous advantages over traditional cell-based assays and in vivo tumor assessments. Therefore, the devices and in vitro methods of the present disclosure can be used for diagnostic and prognostic purposes, research, drug development, drug screening, and personalized medicine.

Additionally, TMM isolated cell lines have the potential to be used for the study of transcriptomic, epigenetic and metabolic changes responsible for increased tumor aggression. Such studies are complicated when carried out in vivo because the establishment of metastatic foci at distal organs significantly alters the expression profile of tumor cells due to the dramatically different conditions at these sites, compared to the original tumor location. Such changes which are a consequence of metastasis rather than causal, often mask gene expression changes underlying increased tumor aggression. Thus, the TMM isolated cell lines can be an ideal system to understand and map genomic and transcriptomic alterations in cancer cells. The latter is of crucial importance since it will not only enhance our understanding of tumor progression, but can also lead to the discovery of new therapeutic targets as well as novel diagnostic and prognostic markers enabling early diagnosis and facilitating efficient treatment.

In light of the disclosure set forth herein, and without limiting the disclosure in any way, in a first aspect of the present disclosure, which may be used with any other aspect, or portion thereof, a TMM device includes a base including a bottom wall and a plurality of base posts extending from the TMM device base, the base posts separated from each other to form spaces between the base posts; and a lid sized to mate with the base, the lid including a mating wall and a plurality of lid posts extending from the mating wall, the lid posts separated from each other to form spaces between the base posts, the lid posts positioned when the lid is mated with the base so as to be able to variably cover the spaces between the base posts based on a rotational position of the lid relative to the base.

In a second aspect of the present disclosure, which may be used with any other aspect, or portion thereof, the base posts form a ring of base posts, and the lid posts form a ring of lid posts, and wherein the ring of lid posts is slightly larger or smaller than the ring of base posts.

In a third aspect of the present disclosure, which may be used with any other aspect, or portion thereof, the rings are concentric rings, or concentric semicircles for the TMM devices 110f and 110G, and wherein the inner or outer diameter of the ring of lid posts is sized to slidingly engage the outer or inner diameter, respectively, of the ring of base posts.

In a fourth aspect of the present disclosure, which may be used with any other aspect, or portion thereof, the base posts and the lid posts have at least substantially the same height.

In a fifth aspect of the present disclosure, which may be used with any other aspect, or portion thereof, the base posts are outer base posts and, wherein the base further includes inner core posts extending from the bottom wall, the inner core posts separated from each other to form spaces between the core posts.

In a sixth aspect of the present disclosure, which may be used with any other aspect, or portion thereof, the inner core posts form a ring, or a semicircle for the TMM devices 110f and 110g.

In a seventh aspect of the present disclosure, which may be used with any other aspect, or portion thereof, the lid and base are configured to releasably fix the rotational position of the lid relative to the base.

In an eighth aspect of the present disclosure, which may be used with any other aspect, or portion thereof, the lid is configured to be fastened to the base to releasably fix the rotational position of the lid relative to the base.

In a ninth aspect of the present disclosure, which may be used with any other aspect, or portion thereof, the mating wall extends from a sealing flange of the lid and a peripheral wall extends from the bottom wall of the base, and wherein the sealing flange is configured to be fastened to the peripheral wall to releasably fix the rotational position of the lid relative to the base.

In a tenth aspect of the present disclosure, which may be used with any other aspect, or portion thereof, wherein the fastening of the lid to the base includes a plurality threaded apertures, each aperture corresponding to a different environmental openness setting.

In an eleventh aspect of the present disclosure, which may be used with any other aspect, or portion thereof, one of the lid or base includes a projection that snap-fittingly mates with one of a plurality of indentations provided by the other of the lid or base to fix the rotational position of the lid relative to the base.

In a twelfth aspect of the present disclosure, which may be used with any other aspect, or portion thereof, each of the indentations corresponds to a different environmental openness setting.

In a thirteenth aspect of the present disclosure, which may be used with any other aspect, or portion thereof, the lid defines at least one media loading aperture for injecting media into a cavity formed between the mating wall of the lid and the bottom wall of the base.

In a fourteenth aspect of the present disclosure, which may be used with any other aspect, or portion thereof, the TMM device further includes a core sealer sized to be placed on the lid, the core sealer including at least one plug that plugs the at least one media loading aperture.

In a fifteenth aspect of the present disclosure, which may be used with any other aspect, or portion thereof, the mating wall extends from a sealing flange of the lid, and wherein the sealing flange defines at least one loading hole for receiving media and/or oxygen radially outside of the spaces and the base posts when the lid is mated with the base.

In a sixteenth aspect of the present disclosure, which may be used with any other aspect, or portion thereof, the TMM device includes at least one media tank sealer for plugging the at least one loading hole.

In a seventeenth aspect of the present disclosure, which may be used with any other aspect, or portion thereof, the TMM device includes at least one divider extending radially from the mating wall and perpendicularly from the sealing flange, the at least one divider blocking a space between the mating wall of the lid and a peripheral wall of the base when the lid is mated to the base.

In an eighteenth aspect of the present disclosure, which may be used with any other aspect, or portion thereof, the lid includes at least one divider extending from the mating wall, the at least one divider enabling different cells to be placed on the bottom wall of the base, the at least one divider enabling the different cells to be separated.

In a nineteenth aspect of the present disclosure, which may be used with any other aspect, or portion thereof, the at least one divider includes two aligned dividers that at least substantially bifurcate the mating wall.

In a twentieth aspect of the present disclosure, which may be used with any other aspect, or portion thereof, any of the features, functionality and alternatives described in connection with any one or more of FIGS. 1 to 27 may be combined with any of the features, functionality and alternatives described in connection with any other of FIGS. 1 to 27.

In a twenty-first aspect of the present disclosure, which may be used with any other aspect, or portion thereof, the method of using any of the TMM devices, 10a to 10h, 110a, and 110d to 110g, to mimic the microenvironment of a tumor and investigate 3D cell invasion and migration, and/or cell responses to various environmental factors or other cells.

In a twenty-second aspect of the present disclosure, which may be used with any other aspect, or portion thereof, the method of using any of the TMM devices, 10a to 10h, 110a, 110d, 110e, 110f, and 110g, to co-culture more than one type of cell and investigate the cell interactions and/or the affect the additional cells may have on the migration or invasion of tumor cells.

In a twenty-third aspect of the present disclosure, which may be used with any other aspect, or portion thereof, the method of adding growth factors to the media located in the media tank via loading holes, or by introducing cell culturing media outside the invasion zones of the device.

In a twenty-fourth aspect of the present disclosure, which may be used with any other aspect, or portion thereof, the method of introducing a selected extracellular matrix (“ECM”) mimic into the invasion zone area of device 10a to 10h, 110a, 110d, 110e, 110f, and 110g, via side openings.

In a twenty-fifth aspect of the present disclosure, which may be used with any other aspect, or portion thereof, the method wherein the cells within the sample core of TMM devices 10a to 10h, 110a, and 110d to 110g consume nutrients and oxygen, and thus, the immediate microenvironment within the sample core mimics a necrotic core or a hypoxic area of a tumor.

In a twenty-sixth aspect of the present disclosure, which may be used with any other aspect, or portion thereof, the method wherein the invasion zone area surrounding the sample core, provides fewer environmental challenges compared to the sample core area, providing a more desirable microenvironment (i.e., the invasion zone area) for the cells located within the sample core.

In a twenty-seventh aspect of the present disclosure, which may be used with any other aspect, or portion thereof, the method wherein regional dividers 46a and 46b extend outwardly from the mating wall 42 and provide a physical separation of the invasion zone, thereby creating more than one distinct invasion zone in any one of the TMM devices 10a to 10g. Further, the regional dividers 46a and 46b can be placed on the TMM device base (22) and extend from the core post (26) outwardly to the outer posts (28) and provide a physical separation of the invasion zone, thereby creating more than one distinct invasion zone in any one of the TMM devices 10a to 10g, 110a, and 110d to 110g.

In a twenty-eighth aspect of the present disclosure, which may be used with any other aspect, or portion thereof, for TMM devices 110f and 110g, the core posts (26) and peripheral posts (28) can be downwards from a base (23) that is extended from the mating wall 33 to provide a physical separation of the core and invasion zone.

In a twenty-ninth aspect of the present disclosure, which may be used with any other aspect, or portion thereof, the method wherein the cells within TMM devices 10a to 10h, 110a, and 110d to 110g can be visualized using microscopy techniques.

In a thirtieth aspect of the present disclosure, which may be used with any other aspect, or portion thereof, the method wherein a tumor cell line of interest is added to the sample core of any of the TMM devices 10a to 10h, 110a, and 110d to 110g and the ability of the primary tumor cells or tumor cell line of interest to migrate or invade the invasion zone may be assessed.

In a thirty-first aspect of the present disclosure, which may be used with any other aspect, or portion thereof, the method to distinguish between metastatic and non-metastatic cell lines.

In a thirty-second aspect of the present disclosure, which may be used with any other aspect, or portion thereof, the method wherein the effects of various environmental factors or cells present within the invasion zone of any of the TMM devices 10a to 10h, 110a, 110d, 110e, 110f, and 110g, on the ability of a tumor cell of interest to migrate or invade the surrounding area is analyzed.

In a thirty-third aspect of the present disclosure, which may be used with any other aspect, or portion thereof, the method of evaluating anti-cancer drugs or other test drugs or compounds by analyzing their effects on the ability of tumor cells to migrate or invade the invasion zone of any of the TMM devices 10a to 10h, 110a, and 110d to 110G in the presence of the test drug or compound.

In a thirty-fourth aspect of the present disclosure, which may be used with any other aspect, or portion thereof, the method of isolating cancer cell subpopulations by introducing tumor cells to the sample core of any of the TMM devices 10a to 10h, 110a, 110d to 110g and allowing them to traverse and exit the invasion zone.

It is accordingly an advantage of the present disclosure to provide a cell culture device that mimics a tumor microenvironment by providing stable growth factor and oxygen gradients, via regulated restriction of nutrients and oxygen intake with the use of the outer posts (28). Additionally, the restriction of nutrients and oxygen intakes can be regulated in any of the TMM devices 10a to 10g by adjusting the lid orientation based on features 58, which affect the positioning of the lid posts (48).

It is another advantage of the present disclosure to provide a low-cost device having a fast and easy protocol for use, yielding simple methods for analysis of cell 3D migration and/or cell invasion, and cancer cell progression.

It is a further advantage of the present disclosure to provide a device and method for analyzing the effectiveness of drugs or compounds on the ability of tumor cells to migrate or invade surrounding areas.

It is still another advantage of the present disclosure to provide a device and method for analyzing the effects of environmental factors, such as oxygen, pressure, cell nutrients, other cells, chemoattractants, and/or growth factors, on the ability of tumor cells to migrate or invade surrounding areas.

It is a further advantage of the present disclosure to provide a device and methods for the in vitro isolation of cancer cell subpopulations which display increased aggressiveness in vivo. These isolated, aggressive cancer cell subpopulations that are generated in the TMM device may be used to further study the tumor cell transcriptomic, epigenetic, and metabolic changes, responsible for tumor progression and increased tumor aggression.

It is a further advantage of the present disclosure to provide a device that through its geometry, cell confinement, and restricted nutrient and oxygen uptake, leads to the establishment of a phenotype that resembles a cross-section of a tumor within 2 days. The microtumor cross-sections established contain the typical zones found in human tumors including a necrotic core, a pre-necrotic zone, a quiescent zone, and a proliferation zone.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1A is a perspective view of a base of a first embodiment for a tumor microenvironment mimicking (“TMM”) device of the present disclosure.

FIG. 1B is a perspective view of a lid for the first embodiment of the TMM device of the present disclosure.

FIG. 1C is a perspective view of a core sealer for the first embodiment of the TMM device of the present disclosure.

FIG. 1D is a perspective view of a second implementation of the base for the first embodiment of the TMM device of the present disclosure.

FIG. 1E is a perspective view of a second implementation of the lid for the first embodiment of the TMM device of the present disclosure.

FIG. 2 is a perspective view of an assembled first embodiment of the TMM device of the present disclosure.

FIG. 3 an exploded, perspective view of the first embodiment of the TMM device of the present disclosure.

FIG. 4 is a first cross-sectional view of an assembled first embodiment of the TMM device of the present disclosure.

FIG. 5 is a second cross-sectional view of an assembled first embodiment of the TMM device of the present disclosure.

FIG. 6 is a third cross-sectional view of an assembled first embodiment of the TMM device of the present disclosure.

FIGS. 7A to 7E are various top plan views of a lid rotated at different positions relative to a base of the assembled first embodiment of the TMM device of the present disclosure.

FIG. 8A is a perspective view of a base of a second embodiment for a TMM device of the present disclosure.

FIG. 8B is a perspective view of a lid for the second embodiment of the TMM device of the present disclosure.

FIG. 8C is a perspective view of a core sealer for the second embodiment of the TMM device of the present disclosure.

FIG. 8D is a perspective view of an assembled second embodiment of the TMM device of the present disclosure.

FIGS. 8E and 8F are first and second cross-sectional views of an assembled second embodiment of the TMM device of the present disclosure.

FIG. 9A is a perspective view of a base of a third embodiment for a TMM device of the present disclosure.

FIG. 9B is a perspective view of a lid for the third embodiment of the TMM device of the present disclosure.

FIG. 9C is a perspective view of a core sealer for the third embodiment of the TMM device of the present disclosure.

FIG. 9D is a perspective view of an assembled third embodiment of the TMM device of the present disclosure.

FIGS. 9E and 9F are first and second cross-sectional views of an assembled third embodiment of the TMM device of the present disclosure.

FIG. 10A is a perspective view of a base of a fourth embodiment for a TMM device of the present disclosure.

FIG. 10B is a perspective view of a lid for the fourth embodiment of the TMM device of the present disclosure.

FIG. 10C is a perspective view of a core sealer for the fourth embodiment of the TMM device of the present disclosure.

FIG. 10D is a perspective view of an assembled fourth embodiment of the TMM device of the present disclosure.

FIGS. 10E and 10F are first and second cross-sectional views of an assembled fourth embodiment of the TMM device of the present disclosure.

FIG. 11A is a perspective view of a base of a fifth embodiment for a TMM device of the present disclosure.

FIG. 11B is a perspective view of a lid for the fifth embodiment of the TMM device of the present disclosure.

FIG. 11C is a perspective view of a core sealer for the fifth embodiment of the TMM device of the present disclosure.

FIG. 11D is a perspective view of an assembled fifth embodiment of the TMM device of the present disclosure.

FIGS. 11E and 11F are first and second cross-sectional views of an assembled fifth embodiment of the TMM device of the present disclosure. FIG. 11G is a perspective view of a sixth embodiment of the TMM device of the present disclosure.

FIGS. 11H and 11I are first and second cross-sectional views of the sixth embodiment of the present disclosure.

FIG. 11J is a perspective view of a seventh embodiment of the TMM device of the present disclosure.

FIG. 11K is a perspective view of an eighth embodiment of the TMM device of the present disclosure.

FIG. 11L is a cross-sectional view of the eighth embodiment of the TMM device of the present disclosure.

FIG. 11M is a disassembled view of a ninth embodiment of the TMM device of the present disclosure.

FIGS. 11N, 11O and 11P are perspective and sectioned assembled views of the ninth embodiment of the TMM device of the present disclosure.

FIGS. 12A and 12B are perspective views of a tenth embodiment of the TMM device of the present disclosure.

FIGS. 13A to 13G are perspective views illustrating one embodiment of a method of the present disclosure for preparing a microtumor assay protocol.

FIG. 14 is a graph illustrating one example invasion of six different cell lines using a TMM device of the present disclosure.

FIGS. 15A to 15F are confocal fluorescent images illustrating another example of a co-culture of cancer cells and fibroblasts cultivated via a TMM device of the present disclosure.

FIGS. 16A, and 16B are confocal fluorescent images illustrating an evaluation of a hypoxia gradient generated after twenty days of culture in the tumor microenvironment mimicking (TMM) device.

FIG. 16C are confocal images illustrating the evaluation of a hypoxia gradient generated 24 hours after uniform introduction of cells within the TMM device.

FIG. 17 is a confocal image illustrating the de-novo established gradient of growth factors towards the center of a TMM device of the present disclosure.

FIG. 18 is a confocal image illustrating a depth-coded reconstruction of cell invasion from the sample core into the invasion zone of a TMM device of the present disclosure.

FIGS. 19A and 19B are perspective views of an eleventh embodiment of the TMM device of the present disclosure.

FIGS. 20A to 20C are perspective views of a twelfth embodiment of the TMM device of the present disclosure.

FIGS. 21A and 21B are perspective views of a twelfth embodiment of the TMM device of the present disclosure in a multiwell configuration.

FIG. 22 are confocal images illustrating the establishment of a necrotic core by three cell lines using a TMM device of the present disclosure.

FIG. 23 is a graph illustrating the pH gradient established 24 hours after the introduction of cells within a TMM device of the present disclosure.

FIG. 24A is a graphical representation of the in vivo tumor growth rates (in nude mice) of the parental cancer cell line (MDA-MB-231) and the subpopulation, which was generated by isolating cells that have traversed and exited the invasion zones of a TMM device of the present disclosure.

FIG. 24B is a table that illustrates the percentage of animals that formed abdominal metastases 9 weeks post injection of nude mice with either the parental cancer cell line (MDA-MB-231) and its subpopulation, which was generated by isolating cells that have traversed and exited the invasion zones of a TMM device of the present disclosure.

FIG. 25 is confocal images illustrating the conversion of normal fibroblasts into cancer associated fibroblasts within the invasion zone of a TMM device of the present disclosure.

FIGS. 26A to 26C are graphical representations of an evaluation of the protein levels of known tumor promoting markers from the parental cancer cell line and its subpopulation, which was generated using a TMM device of the present disclosure.

FIGS. 27A to 27E are perspective views of a thirteenth embodiment of the TMM device of the present disclosure as a two tiered multi-well insert.

### DETAILED DESCRIPTION

### Non-Limiting Definitions

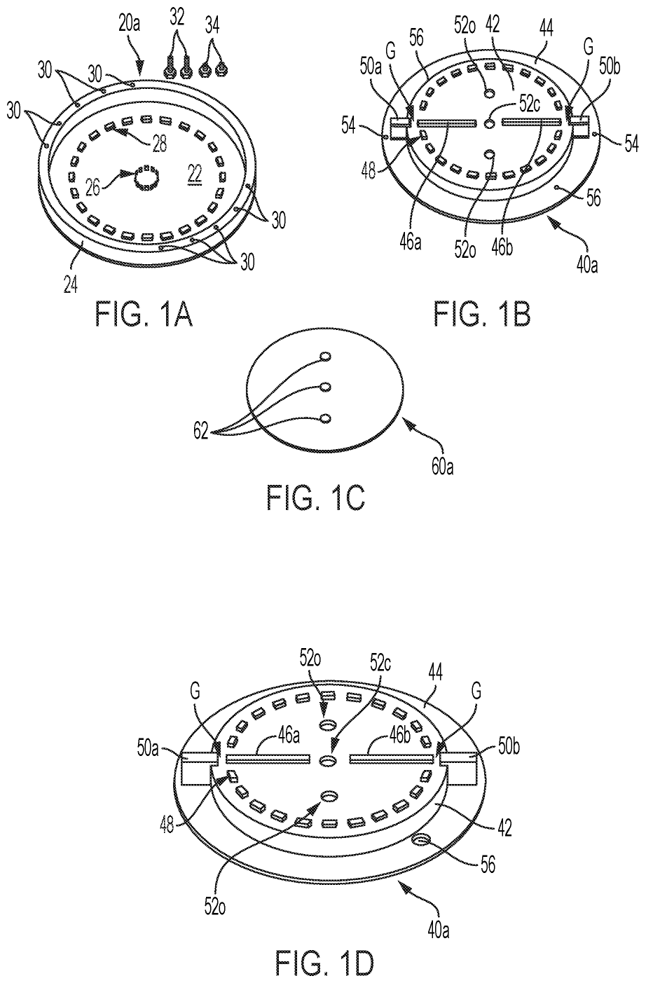

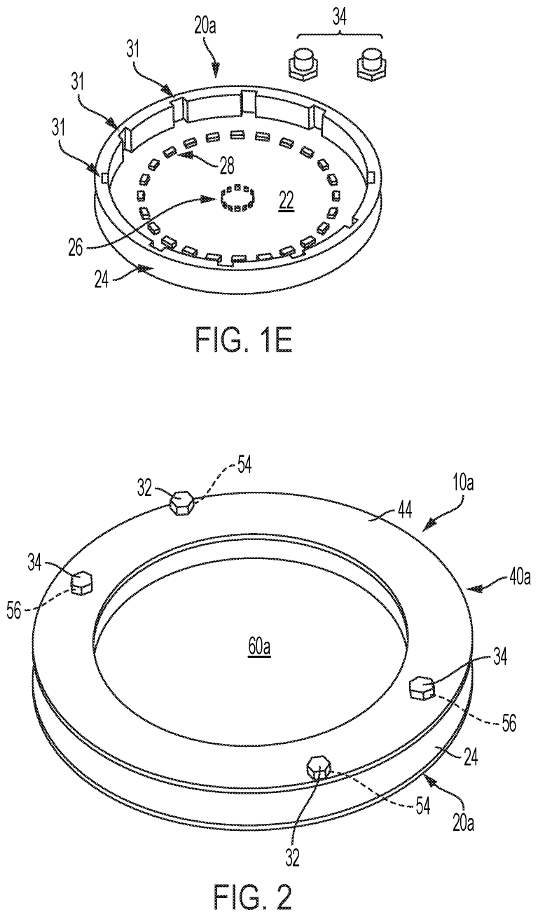

Referring to FIGS. 1A to 11F, 19A to 21B, and 27A to 27E, the terms “sample core area” and “sample core” may be used herein to describe the center area of a bottom wall 22 of a base 20a of any one of devices 10a to 10g, 110a, 110d, 110e, 110f, and 110g, with an outer boundary of the “sample core area” or “sample core” physically marked by an inner ring of core posts 26.

The terms “invasion zone area” and “invasion zone” may be used herein to describe an area on bottom wall 22 of the base 20a, which is peripheral to or adjacent to the sample core, the area bounded by the inner ring of core posts 26 and an outer ring of peripheral posts 28 of any one of devices 10a to 10g, 110a, and 110d to 110g.

The terms “media tank” or “media reservoir” may be used herein to describe a volume provided at an area between an inner surface of cylindrical peripheral wall 24 and circular inner surface 22 of the base 20a and an inner mating wall 42 of the lid 40a.

### TMM Devices

Referring now to the drawings and in particular to FIGS. 1A to 7E, an embodiment of a tumor microenvironment mimicking (“TMM”) device 10a and associated methodology is illustrated. TMM device 10a and any of alternative TMM devices 10b to 10h may be formed in whole or in part from one or more biocompatible metal, such as, titanium and titanium alloys, stainless steel, NI—Ti alloy (Nitinol™), and/or a cobalt-chromium alloy. TMM devices 10a to 10h, 110a, 110d, 110e, 110f, and 110g may alternatively or additionally be formed in whole or in part from one or more ceramic biomaterial, such as, alumina/aluminosilicates, bioglass and/or A-W glass ceramic, hydroxyapatite, zirconia, tricalcium phosphate, carbon/carbon filters/pyrolytic carbon (LTI)/vitreous carbon, carbides, calcium phosphate, and/or graphite.

TMM devices 10a to 10h, 110a, and 110d to 110g may alternatively or additionally be formed in whole or in part from one or more polymer, such as, polydimethylsiloxane (“PDMS”), resin/phenol formaldehyde resins/melamine resins/polyepoxides, polyvinylchloride (“PVC”), polyvinyl alcohol (“PVOH”), polyvinyl acetate (“PVAC”), polyvinylidene chloride (“PVDC”), polyvinyl butryal (“PVB”), polyethylene (“PE”), polypropylene (“PP”), polymethylmetacrylate (“PMMA”), polystyrene (“PS”), polytetrafluoroethylene (“PTFE”), polyurethane (“PU”) and polyamide (nylon), polyethylenterephthalate (“PET”), polyethersulfone (“PES”), polyetherimide (“PEI”), medical grade silicone, TMC NAD-lactide, polylactic acid (PLA), cyclic olefin copolymer (“COC”), polycarbonate (“PC”), polyetheretherketone (“PEEK”), poly(lactic-co-glycolic acid) (“PLGA”), acrylonitrile-butadiene-styrene (“ABS”), and other styrene copolymers (SAN, MBS, SBS, SIS), acetal-poloyxymethylene (“POM”), ethylene vinyl acetate copolymers (“EVA”), polyacrilonitrile (“PAN”), celluloid acetate (“CA”), polybutylene terephthalate (“PBT”), polytetramethylene terephthalate (“PTMT”), polytetrafluorethylene (“PTFE”), polychlorotrifluorethylene (“PCTFE”), polyvinyl fluoride (“PVDF”), polyimides (“PI”), and/or trimethylcarbonate.

Any components of devices 10a to 10h, 110a, and 110d to 110g made of a biocompatible metal may be machined or formed. Any components of devices 10a to 10h, 110a, 110d, 110e, 110f, and 110g made of a ceramic biomaterial may likewise be machined or formed. Any components of devices 10a to 10h, 110a, and 110d to 110g made of a polymer may be molded, e.g., injection molded or may be made of a three-dimensional printing or additive manufacturing process.

FIG. 1A illustrates that TMM device 10a includes a base 20a. Base 20a includes a bottom wall 22 from which a peripheral wall 24 extends. In the illustrated embodiment, bottom wall 22 is circular, while peripheral wall 24 is cylindrical. Bottom wall 22 in the illustrated embodiment includes an inner ring of core posts 26 and an outer ring of peripheral posts 28. The rings of posts 26 and 28 are circular in one embodiment. Posts 26 and 28 may each extend perpendicularly from bottom wall 22 and have inner and outer curved or radiused surfaces. Posts 26 in the illustrated embodiment are the same height, but smaller in diameter than posts 28.

In the illustrated embodiment, the top of peripheral wall 24 is provided with mounting holes 30, which allow a lid 40a of TMM device 10a to be mounted in a selected and desired orientation relative to base 20a. Mounting holes 30 may be threaded to threadingly receive threaded bolts 32 illustrated in FIG. 1A. FIG. 1A also illustrates that TMM device 10a includes media tank sealers 34, which are discussed in detail below. Threaded bolts 32 and media tank sealers 34 may be made of any of the materials discussed herein.

FIG. 1B illustrates that TMM device 10a includes a lid 40a that adjustably mounts to base 20a. FIG. 1B illustrates the more detailed underside of lid 40a. Lid 40a is flipped 180 degrees from the position shown in FIG. 1B for mounting to base 20a. In the illustrated embodiment, lid 40a includes an inner mating wall 42 that is raised or offset from an outer sealing flange 44. Similar to posts 26 and 28 of base 20a, inner mating wall 42 is provided with a ring of posts 48 that extend out from the mating wall. Posts 48 may each extend perpendicularly from inner mating wall 42 and have inner and outer curved or radiused surfaces. The height of posts 48 may be the same as the height of posts 28 of base 20a.

In an embodiment, the height of peripheral wall 24 of base 20a, the amount that mating wall 42 that is raised or offset from an outer sealing flange 44, and the heights of posts 28 and 48 are selected such that when lid 40a is mounted to base 20a, (i) the tops of posts 28 slidingly mate with the mating surface of mating wall 42 and (ii) the tops of posts 48 slidingly mate with the mating surface of bottom wall 22 of base 20a. Also, and as illustrated below, the rings of posts 28 and 48 may have radii or diameters sized such that the outer curved or radiused surfaces of posts 48 slidingly mate with the inner curved or radiused surfaces of posts 28.

In the illustrated embodiment, regional dividers 46a and 46b extend upwardly from mating wall 42. Regional dividers 46a and 46b may be generally rectangular in length and have a height the same as posts 48. Regional dividers 46a and 46b, as illustrated below, may also be sized so as to extend inwardly so as to slidingly mate with (or come very close to) the outer curved or radiused surfaces of posts 26 when lid 40a is mounted to base 20a.

Environmental dividers 50a and 50b extend from or are attached to the side of mating wall 42. Environmental dividers 50a and 50b are spaced apart from the outer edges of regional dividers 46a and 46b, respectively, by a gap G. Gaps G are sized and located so as to receive posts 28 of base 20a as the user twists lid 40a into a desired position relative to the base. Environmental dividers 50a and 50b also provide barriers between mating wall 42 of lid 40a and peripheral wall 24 of base 20a when the lid is mounted to the base. Environmental dividers 50a and 50b also regulate the intake of media and ambient air through the small cut in the mating wall 42.

In the illustrated embodiment, mating wall 42 forms or defines media loading apertures 520 and 52c for injecting media (as discussed in detail below) into the cavity formed between mating wall 42 and bottom wall 22 when lid 40a is mounted to base 20a.

In the illustrated embodiment, outer sealing flange 44 forms or defines mounting holes 54 and loading holes 56. Mounting holes 54 are spaced apart so as to align with threaded mounting holes 30 formed or defined in peripheral wall 24 of base 20a. As illustrated in FIG. 2, after the user aligns mounting holes 54 with a desired set of mounting holes 30, the user fixes lid 40a to base 20a by inserting threaded bolts 32 through mounting holes 54 and threading the bolts tight into threaded mounting holes 30.

Loading holes 56 are spaced closer together than are mounting holes 54. Loading holes 56 are spaced so as to provide selective access into the space between the inner surface of peripheral wall 24 and the outer surface of mating wall 42 when lid 40a is mounted to base 20a. The purpose for the selective access into that space is to allow media and oxygen to enter TMM device 10a as discussed in more detail below. As illustrated in FIG. 2, when selective access into that space is not needed, loading holes 56 are plugged via media tank sealers 34.

FIG. 1C illustrates that TMM device 10a further includes a core sealer 60a that the user places into the top side of lid 40a (as illustrated in FIG. 2) after the user has loaded any desired media into the cavity formed between mating wall 42 and bottom wall 22 when lid 40a is mounted to base 20a. FIG. 1C illustrates the more detailed underside of core sealer 60a. Core sealer 60a is flipped 180 degrees from the position shown in FIG. 1C for placement onto mating wall 42 of lid 40a. In the illustrated embodiment, the underside of core sealer 60a forms or defines plugs 62 that plug loading apertures 520 and 52c of mating wall 42 of lid 40a. In this manner, the cavity formed between mating wall 42 and bottom wall 22 when lid 40a is mounted to base 20a is sealed relative to the ambient world when core sealer 60a is placed onto mating wall 42 of lid 40a.

FIG. 1D illustrates a second implementation of lid 40a, in which environmental dividers 50a and 50b extend further from the mating wall 42, such that the environmental dividers 50a and 50b engage the notches 31 in the peripheral wall 24 of the base 20a in a selected and desired orientation. It should be appreciated that the second implementation of lid 40a does not include mounting holes 54 or threaded bolts 34. It should also be appreciated that without mounting holes 54 or threaded bolts 34, the second implementation of the lid 40a could fasten to a second implementation of base 20a with an alternative locking mechanism, such as a snap fit or with mounting bosses.

FIG. 1E illustrates a second implementation of base 20a, in which peripheral wall 24 includes notches 31, which allows lid 40a of TMM device 10a to engage the base 20a in a selected and desired orientation. The thickness of the peripheral wall 24 is such that notches 31 do not extend therethrough.

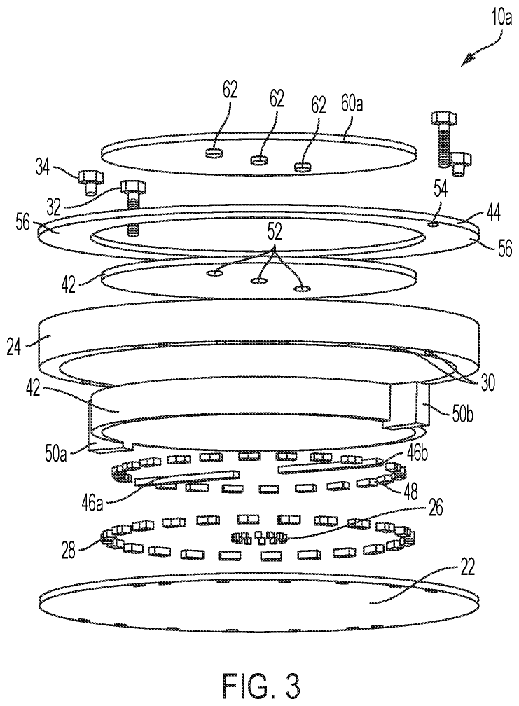

Although the following text describes device 10a using the screw embodiment as shown in FIGS. 1A and 1B, the engagement between notches 31 and extended environmental dividers 50a and 50b can be applied as an alternative according to FIGS. 1D and 1E. Such engagement may be provided in combination with any and all of the text associated with FIGS. 3 to 7E.

FIG. 3 shows the components of TMM device 10a just described in an exploded view to give additional spatial detail. Starting from the top, TMM device 10a includes core sealer 60a having plugs 62 that plug loading apertures 520 and 52c of mating wall 42. Threaded bolts 32 extend through mounting holes 54 formed in the outer sealing flange 44 and thread into threaded mounting holes 30 formed or defined in peripheral wall 24. Tank sealers 34 selectively plug loading holes 56 formed in the outer sealing flange 44 when desired by the user. Peripheral wall 24 extends around the cylindrical side of mating wall 42. Environmental dividers 50a and 50b provide barriers between the side of mating wall 42 and the peripheral wall 24. Regional dividers 46a and 46b and the ring of posts 48 are formed with or attached to the underside of mating wall 42. Inner ring of core posts 26 and the outer ring of peripheral posts 28 are formed with or attached to the topside of bottom wall 22. The ring of posts 48 fits slidingly inside the ring of peripheral posts 28.

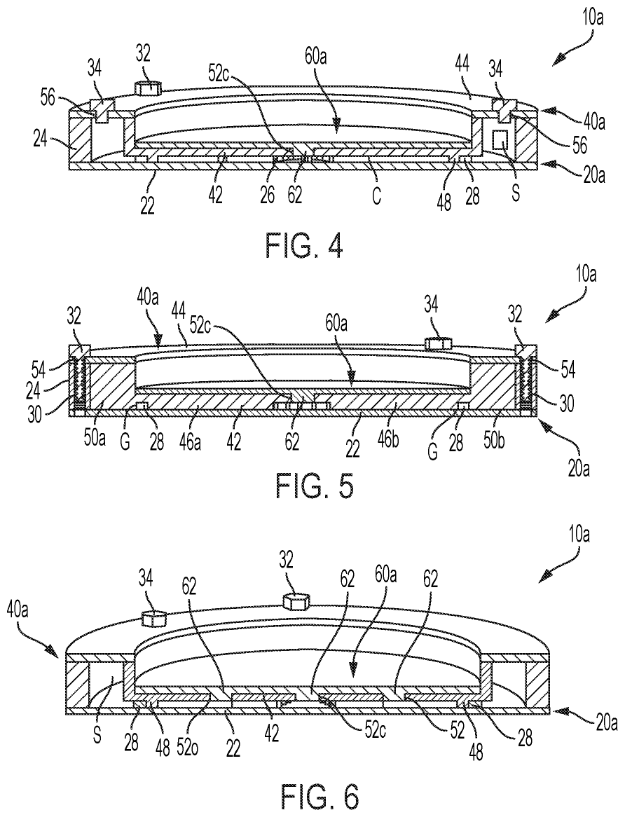

FIG. 4 is a cross-sectional view of TMM device 10a highlighting certain interactions between base 20a, lid 40a and core sealer 60a. In particular, FIG. 4 illustrates media tank sealers 34 selectively filling or plugging loading holes 56 so as to close off and seal a space S between peripheral wall 24 and the sidewall of mating wall 42. Also, one of the plugs 62 of core sealer 60a is illustrated as filling or plugging one of media loading apertures 52c of mating wall 42. Additionally, posts 48 of mating wall 42 of lid 40a are shown slidingly engaging peripheral posts 28 of bottom wall 22 of base 20a. Moreover, mating wall 42 and bottom wall 22 are illustrated as forming a cavity C within which media grows as discussed in detail below.

FIG. 5 is a different cross-sectional view of TMM device 10a highlighting additional interactions between base 20a, lid 40a and core sealer 60a. In particular, FIG. 5 illustrates threaded bolts 32 extending through mounting holes 54 formed in outer sealing flange 44 of lid 40a and threading into threaded mounting holes 30 formed in peripheral wall 24 of base 20a. Again, one of the plugs 62 of core sealer 60a is illustrated as filling or plugging one of media loading apertures 52c of mating wall 42. Additionally, environmental dividers 50a and 50b are illustrated as blocking the space S in FIG. 4 between peripheral wall 24 and the sidewall of mating wall 42. Moreover, gaps G formed between regional dividers 46a and 46b and environmental dividers 50a and 50b, respectively, are illustrated as being slidingly filled by posts 28 of bottom wall 22 of base 20a.

FIG. 6 is yet another cross-sectional view of TMM device 10a highlighting further additional interactions between base 20a, lid 40a and core sealer 60a. In particular, FIG. 6 illustrates all three plugs 62 of core sealer 60a filling or plugging all three loading apertures 520 and 52c of mating wall 42. Additionally, posts 48 of mating wall 42 of lid 40a are shown slidingly engaging peripheral posts 28 of bottom wall 22 of base 20a.

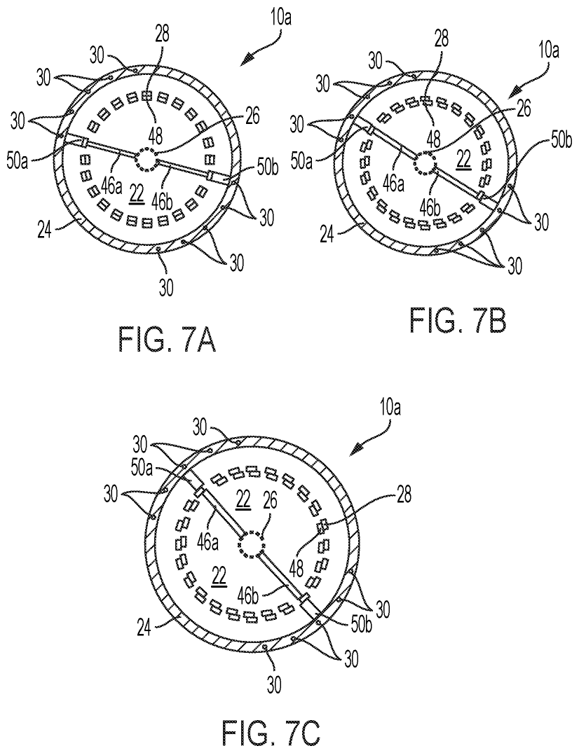

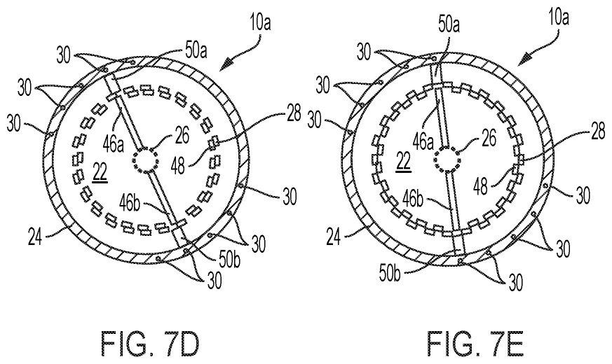

FIGS. 7A to 7E illustrate the five different mounting or engagement settings possible between lid 40a and base 20a. While five settings are illustrated, more or less than five settings may be provided. The settings are defined by 180 degree pairs of threaded mounting holes 30. The user rotates lid 40a so that mounting holes 54 of outer sealing flange 44 (not illustrated in FIGS. 7A to 7E) become aligned with a desired pair of threaded mounting holes 30 and then bolts lid 40a in place to base 20a using threaded bolts 32. The selection of one of the pairs of threaded mounting holes 30 is made based on how open the user wants the cell culture to be to the oxygen and/or media. Media and/or oxygen are provided in space S, for example, illustrated in FIGS. 4 and 6, which may be opened to ambient atmosphere by removing media tank sealers 34 from loading holes 56 formed in outer sealing flange 44 of lid 40a.

The amount that TMM device 10a is open is dictated by the relative slideable position between ring of posts 48 of lid 40a and ring of posts 28 of base 20a. In FIGS. 7A to 7E, core sealer 60a is removed completely, while mating wall 42 and outer sealing flange 44 of lid 40a are removed to show the interaction of posts 48 with posts 28 provided by or attached to bottom wall 22 of base 20a. Regional dividers 46a and 46b and environmental dividers 50a and 50b of lid 40a are also shown and indicate the user's selection because that are aligned with mounting holes 54 of lid 40a, which mate with threaded mounting holes 30 of base 20a.

In FIG. 7A, the user has selected to mount lid 40a to the furthest counterclockwise-most pair of threaded mounting holes 30, which corresponds to posts 48 and posts 28 being fully aligned, providing the most media and/or oxygen in an “environmentally-open” setting. In FIG. 7B, the user has selected to mount lid 40a to the second furthest counterclockwise-most pair of threaded mounting holes 30, which corresponds to posts 48 and posts 28 being slightly offset, providing the second most “environmentally-open” setting. In FIG. 7C, the user has selected to mount lid 40a to the middle pair of threaded mounting holes 30, which corresponds to posts 48 and posts 28 being roughly halfway offset, providing a middle “environmentally-open” setting. In FIG. 7D, the user has selected to mount lid 40a to the second furthest clockwise-most pair of threaded mounting holes 30, which corresponds to posts 48 and posts 28 being mostly offset, providing the second most “environmentally-closed” setting. In FIG. 7E, the user has selected to mount lid 40a to the furthest clockwise-most pair of threaded mounting holes 30, which corresponds to posts 48 and posts 28 being fully offset, providing the most “environmentally-closed” setting.

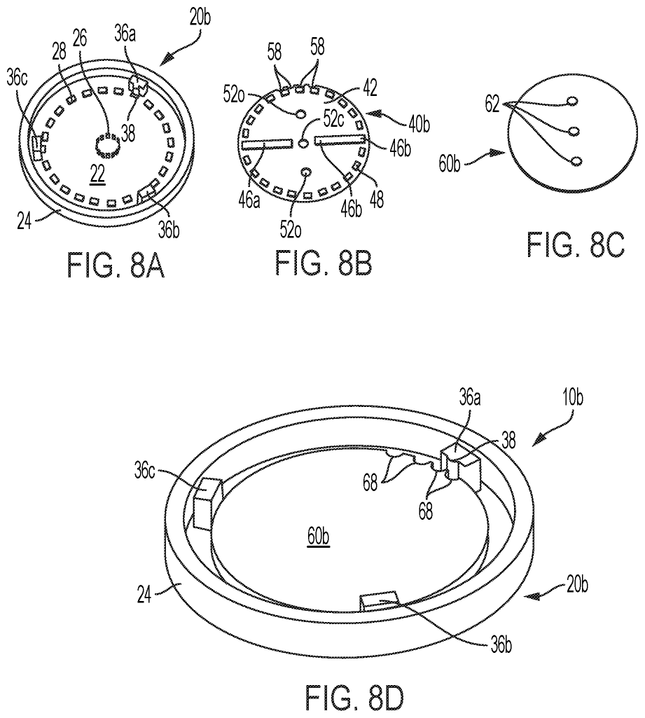

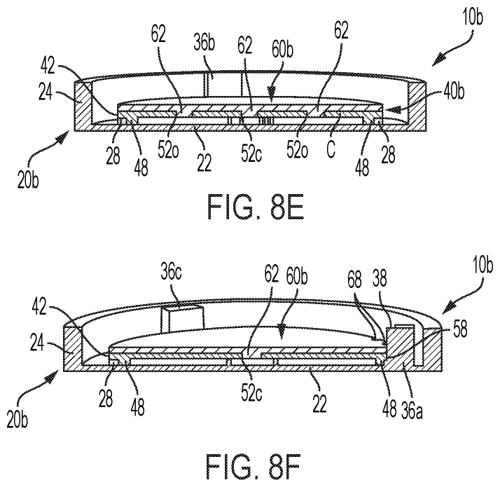

Referring now to FIGS. 8A to 8F, an alternative TMM device 10b having an alternative between base 20b, lid 40b and core sealer 60b is illustrated, which may be made of any of the materials described above. Alternative TMM device 10b includes many of the same components as TMM device 10a, which are numbered the same and include all structure, features and alternatives described above for those components. The primary difference with TMM device 10b is how the “environmental openness” setting is made and how media/or and oxygen are allowed to reach the injected medium.

Lid 40b of TMM device 10b is not provided with an outer sealing flange 44 and is not bolted to base 20b and is generally flat, like core sealer 60b. Peripheral wall 24 of base 20b is accordingly not provided with threaded mounting holes 30. Bottom wall 22 of base includes inner ring of core posts 26 and outer ring of peripheral posts 28 as before. Lid 40b provides regional dividers 46a and 46b and a ring of posts 48 as before. Environmental dividers 50a and 50b are not provided with lid 40b.

Base 20b includes guides 36a to 36c, which are formed with or attach to bottom wall 22 and extend up from the bottom wall. Guides 36a to 36c are spaced apart a distance roughly equal to the diameter of mating wall 42 of lid 40b. Guides 36a to 36c accordingly center lid 40b when placed on base 20b so that posts 48 slidingly engage peripheral posts 28 as with TMM device 10a. The varying engagement of posts 48 and posts 28 (fully aligned to fully offset) varies the “environmental openness” of media and/or oxygen for TMM device 10b in the same manner as described with TMM device 10a.

Guide 36a is formed with or is attached to a projection 38. Mating wall 42 is formed with a plurality of like-shaped indentations 58, for example, three to five indentations, which correspond to the “environmental openness” settings in a similar manner as do threaded holes 30 of TMM device 10b. Indentations 58 snap-fit onto projection 38 of guide 36a to temporarily hold lid 40b in a desired position (“environmental openness” setting) relative to base 20b until the user desires to change the setting and rotate lid 40b. If desired, guides 36b and/or 36c may also include projections 38 for mating with their own sets of indentations 58 formed in mating wall 42. Unlike TMM device 10a, media and/or oxygen are free to reach posts 28 and 48. With TMM device 10b, there is no secondary space S (FIGS. 4 and 6) which can be sealed and unsealed via media tank sealers 34.

FIG. 8D shows an assembled TMM device 10b. Core sealer 60b is substantially the same as core sealer 60a and includes plugs 62 that plug loading apertures 520 and 52c of mating wall 42 of lid 40a. Core sealer 60b may be formed with a smaller diameter than mating wall 42 (FIG. 8C) so as to not interfere with projection 38 of guide 36a. Or, as illustrated in FIG. 8D (and FIG. 8F), core sealer 60b may be formed with the same diameter as mating wall 42 and include its own indentations 68, e.g., shaped the same as indentations 58, which likewise snap-fit onto projection 38 of guide 36a to additionally temporarily hold lid 40b in place. In a further alternative embodiment, indentations 58 in mating wall 42 are not provided, and only indentations 68 in core sealer 60b are provided. Rotating core sealer 60b also rotates lid 40a, which are in mechanical communication via plugs 62 of core sealer 60b and loading apertures 520 and 52c of mating wall 42.

FIG. 8E is a cross-sectional view of TMM device 10b highlighting certain interactions between base 20b, lid 40b and core sealer 60b. In particular, FIG. 8E illustrates all three plugs 62 of core sealer 60b filling or plugging all three loading apertures 520 and 52c of mating wall 42. Additionally, posts 48 of mating wall 42 of lid 40b are shown slidingly engaging peripheral posts 28 of bottom wall 22 of base 20b. FIG. 8E also illustrates cavity C residing between mating wall 42 and bottom wall 22 within which media grows as discussed in detail below.

FIG. 8F is a different cross-sectional view of TMM device 10b highlighting additional interactions between base 20b, lid 40b and core sealer 60b. In particular, FIG. 8F illustrates one of indentations 58 snap-fitting onto and interfacing with projection 38 of guide 36a to hold lid 40b temporarily in place. FIG. 8F also shows one of plugs 62 of core sealer 60b filling or plugging a loading aperture 52c of mating wall 42. Additionally, a post 48 of mating wall 42 of lid 40b is shown slidingly engaging a peripheral post 28 of bottom wall 22 of base 20b.

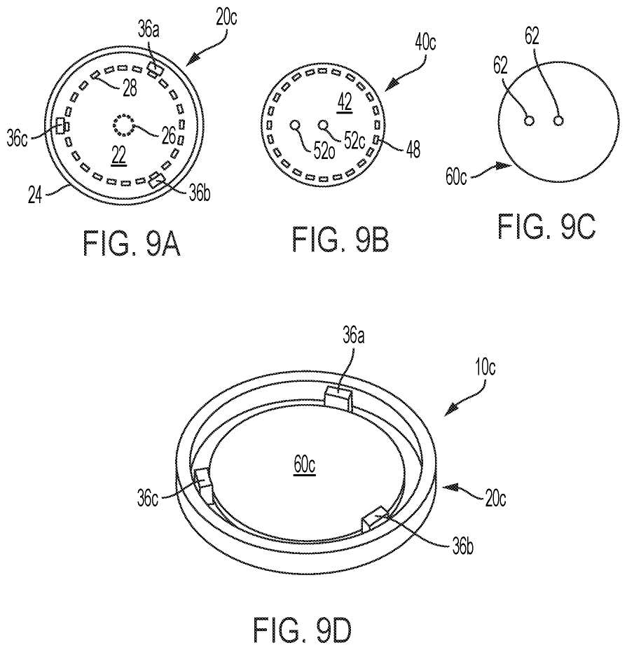

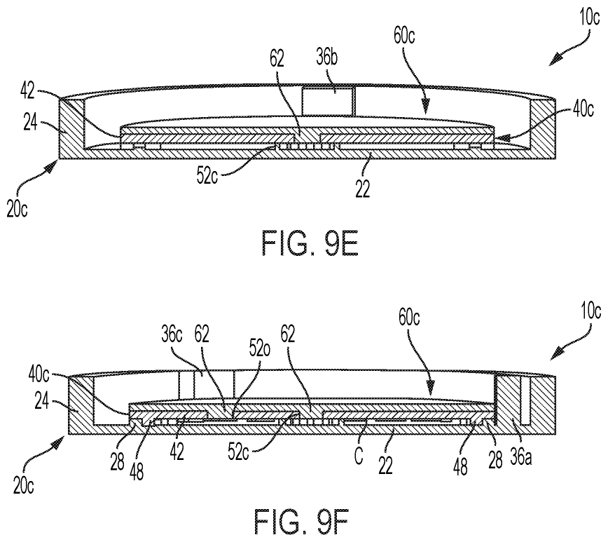

Referring now to FIGS. 9A to 9F, another alternative TMM device 10c having an alternative between base 20c, lid 40c and core sealer 60c is illustrated, which may be made of any of the materials described above. Alternative TMM device 10c includes many of the same components as TMM device 10b, which are numbered the same and include all structure, features and alternatives described above for those components. Guides 36a to 36c again center lid 40c when placed on base 20c so that posts 48 slidingly engage peripheral posts 28 as with TMM device 10b. The varying engagement of posts 48 and posts 28 (fully aligned to fully offset) varies the “environmental openness” of media and/or oxygen for TMM device 10c in the same manner as described with TMM device 10a. Also, like with TMM device 10b, media and/or oxygen are free to reach posts 28 and 48. With TMM device 10c, there is no secondary space S (FIGS. 4 and 6) which can be sealed and unsealed via media tank sealers 34. The primary difference between TMM device 10c and TMM device 10b is how lid 40c is held in place and that regional dividers 46a and 46b are not provided.

FIG. 9A illustrates that base 20c is the same as base 20b except that guide 36a does not include a projection 38. Lid 40c in FIG. 9B accordingly does not include any indentations 58, while core sealer 60c in FIGS. 9C and 9D accordingly does not include any indentations 68. Instead, the diameter of mating wall 42 may be sized to press-fit within guides 36a to 36c of base 20c, such that lid 40c is held temporarily in place when pressed between guides 36a to 36c until the user decides to rotate lid 40c relative to base 20c.

Regional dividers 46a and 46b in TMM devices 10a and 10b allow different media to be injected and present at the same time within the cavities C of the TMM devices. The lack of regional dividers 46a and 46b in TMM device 10c means that only a single cell type is allowed to be injected into cavity C of the TMM device at a given time. Accordingly, only two media loading apertures 520 and 52c need to be formed in mating wall 42 and only two corresponding plugs 62 need to be provided by core sealer 60b.

FIGS. 9E and 9F are cross-sectional views of TMM device 10c highlighting certain interactions between base 20c, lid 40c and core sealer 60c. In particular, FIG. 9E illustrates central plug 62 of core sealer 60c filling or plugging central loading aperture 52c of mating wall 42. FIG. 9F illustrates both plugs 62 of core sealer 60c filling or plugging both loading apertures 520 and 52c of mating wall 42. Additionally, posts 48 of mating wall 42 of lid 40c are shown slidingly engaging peripheral posts 28 of bottom wall 22 of base 20c. Lid 40c and core sealer 60c are also illustrated, fitting snugly against guide 36a (and do so likewise against guides 36b and 36c). FIG. 9F further illustrates cavity C residing between mating wall 42 and bottom wall 22 within which contains the media and oxygen as described in detail below.

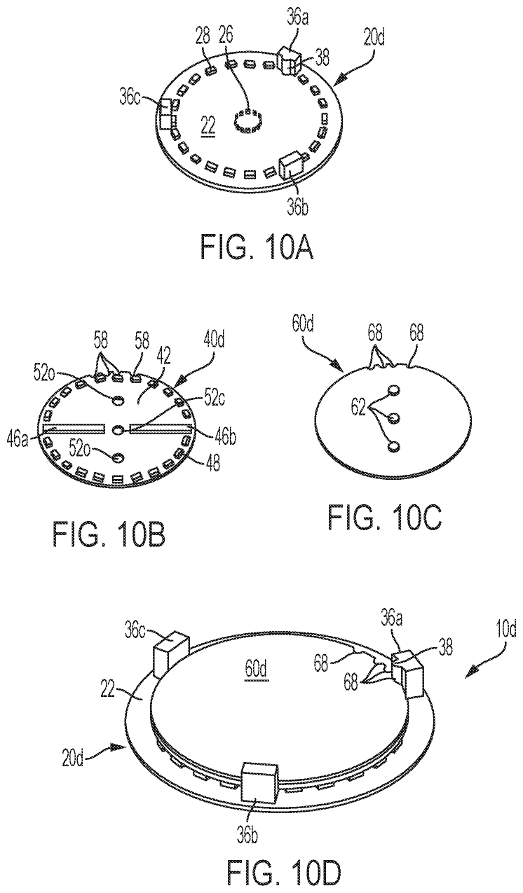

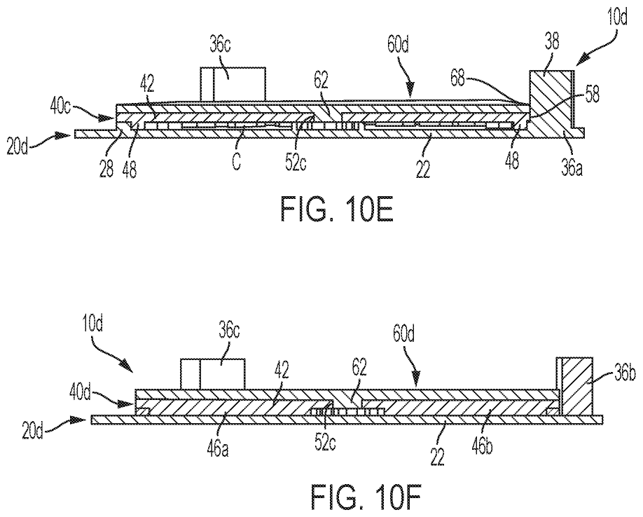

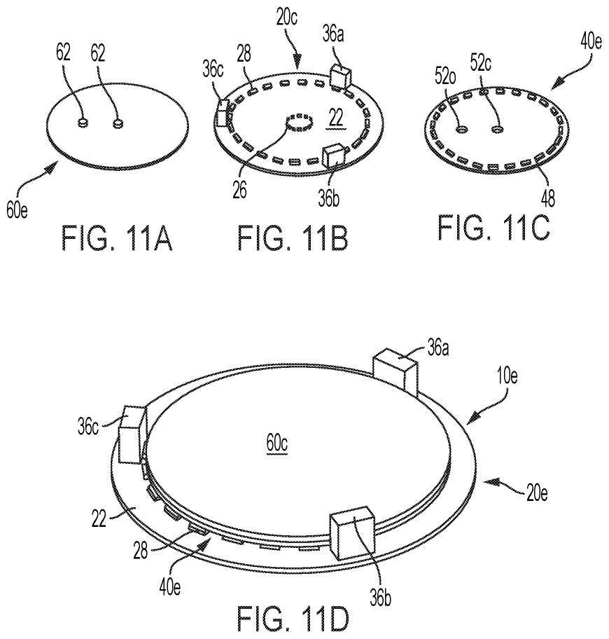

Referring now to FIGS. 10A to 10F, a further alternative TMM device 10d having an alternative between base 20d, lid 40d and core sealer 60d is illustrated, which may be made of any of the materials described above. As illustrated best in FIGS. 10A to 10C, further alternative TMM device 10d is the same device as TMM device 10b (FIGS. 8A to 8F) except that peripheral wall 24 of base 20d is not provided. All other common components are numbered the same and operate the same, including all alternatives. Guides 36a to 36c again center lid 40d when placed on base 20d so that posts 48 slidingly engage peripheral posts 28 as with TMM device 10b. The varying engagement of posts 48 and posts 28 (fully aligned to fully offset) varies the “environmental openness” of media and/or oxygen for TMM device 10d in the same manner as described with TMM device 10a. Also, like with TMM devices 10b and 10c, media and/or oxygen are free to reach posts 28 and 48, especially here with peripheral wall 24 removed.

As with TMM device 10b, indentations 58 in lid 40d snap-fit onto projection 38 of guide 36a of TMM device 10d to temporarily hold lid 40d in a desired position (“environmental openness” setting) relative to base 20d until the user desires to change the setting and rotate lid 40d. Likewise, as illustrated in FIGS. 10C and 10D, indentations 68 in core sealer 60d snap-fit onto projection 38 of guide 36a of TMM device 10d to temporarily hold lid 40d in a desired position (“environmental openness” setting) relative to base 20d until the user desires to change the setting and rotate lid 40d.

FIGS. 10E and 10F are cross-sectional views of TMM device 10d highlighting certain interactions between base 20d, lid 40d and core sealer 60d. In particular, FIG. 10E (and FIG. 10F) illustrates central plug 62 of core sealer 60d filling or plugging central loading aperture 52c of mating wall 42. FIG. 10E also illustrates one of indentations 58 and 68 snap-fitting onto and interfacing with projection 38 of guide 36a to hold lid 40d temporarily in place. Additionally, a post 48 of mating wall 42 of lid 40d is shown slidingly engaging a peripheral post 28 of bottom wall 22 of base 20d. FIG. 10E further illustrates cavity C residing between mating wall 42 and bottom wall 22 within which media grows as discussed in detail below. FIG. 10F also illustrates sectioned regional dividers 46a and 46b, which allow different media to be cultivated at the same time on different sides of the dividers.

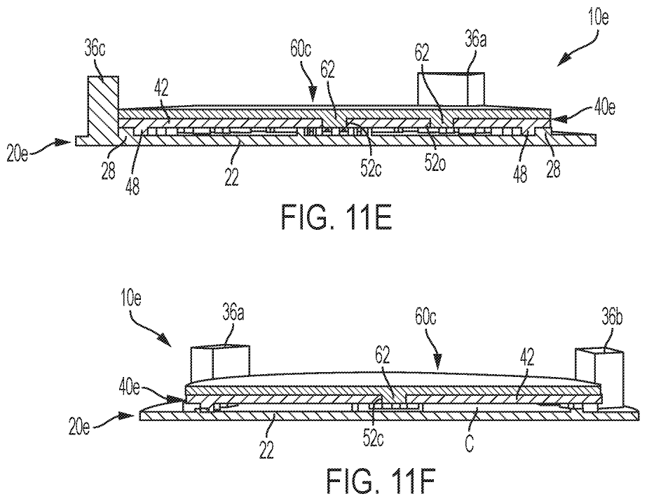

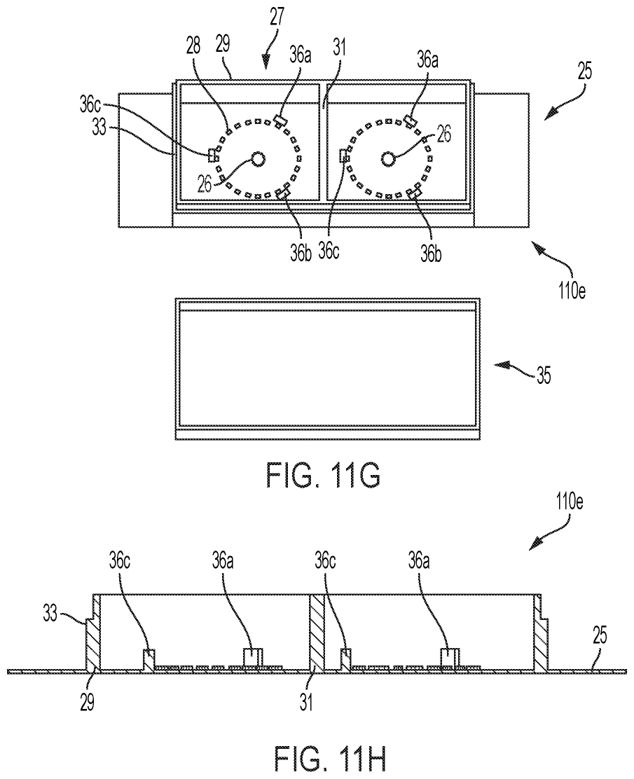

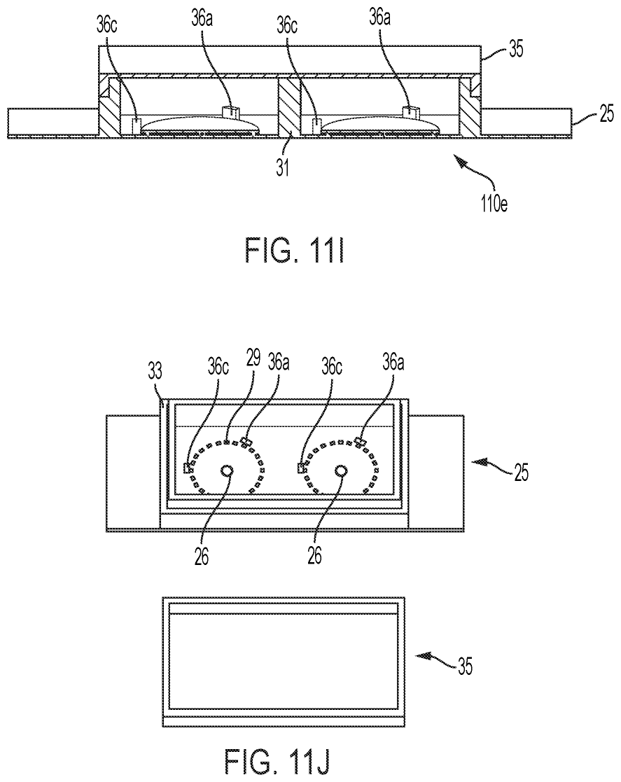

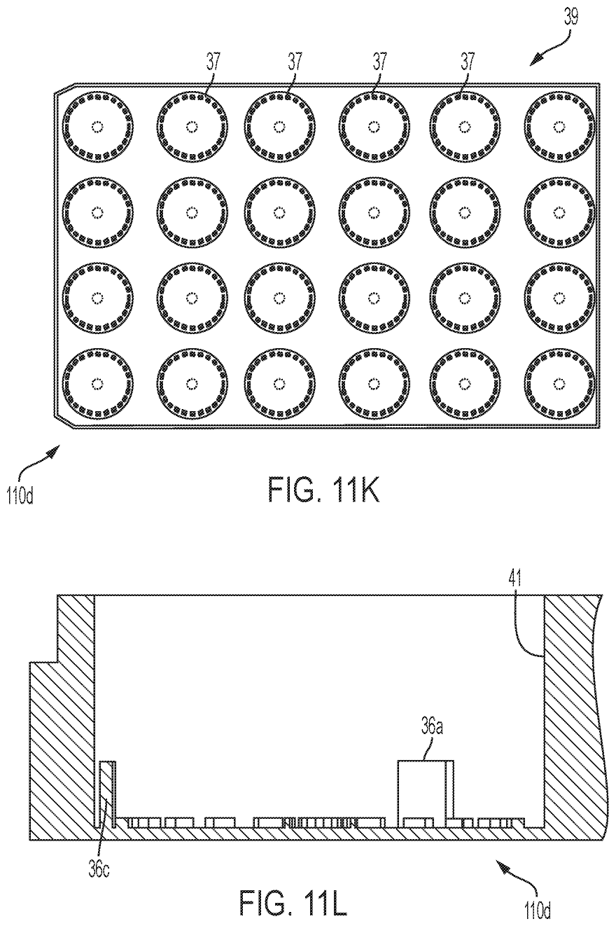

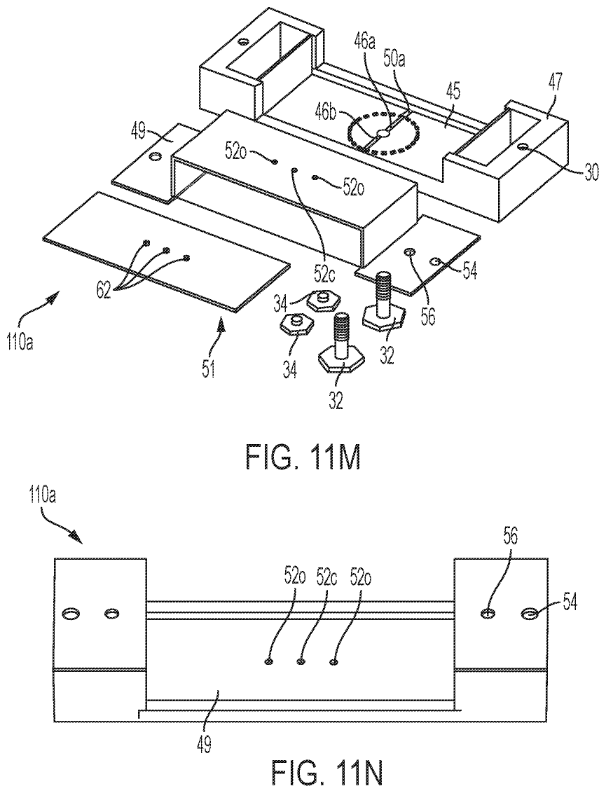

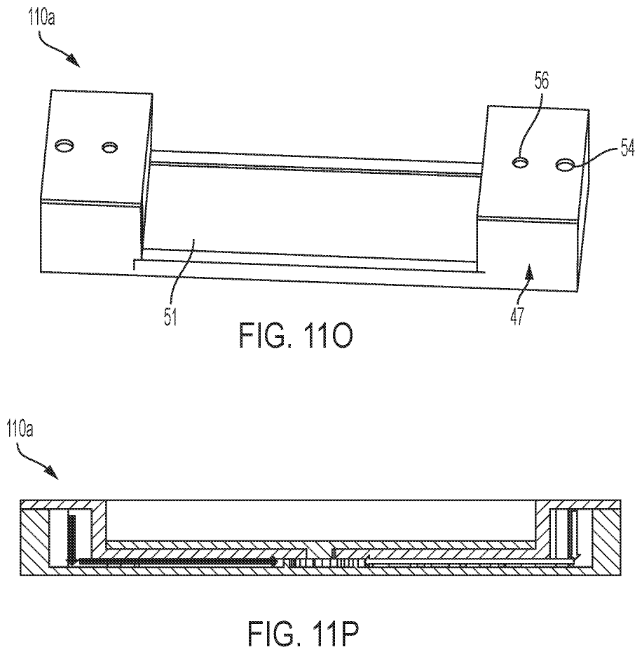

Referring now to FIGS. 11A to 11F, yet another alternative TMM device 10e having an alternative between base 20e, lid 40e and core sealer 60e is illustrated, which may be made of any of the materials described above. As illustrated best in FIGS. 11A to 11C, still further alternative TMM device 10e is the same device as TMM device 10c (FIGS. 9A to 9F) except that peripheral wall 24 of base 20e is not provided. All other common components are numbered the same and operate the same, including all alternatives. Guides 36a to 36c again center lid 40d when placed on base 20d so that posts 48 slidingly engage peripheral posts 28 as with TMM device 10b. The varying engagement of posts 48 and posts 28 (fully aligned to fully offset) varies the “environmental openness” of media and/or oxygen for TMM device 10d in the same manner as described with TMM device 10a. Also, like with TMM devices 10b to 10d, media and/or oxygen are free to reach posts 28 and 48, especially here with peripheral wall 24 removed.

FIG. 11A illustrates that base 20e, like base 20c, includes guides 36a to 36c but does not include a projection 38. Lid 40e in FIG. 11B accordingly does not include any indentations 58, while core sealer 60c in FIGS. 11C and 11D accordingly does not include any indentations 68. Instead, the diameter of mating wall 42 may be sized to press-fit within guides 36a to 36c of base 20c, such that lid 40e is held temporarily in place when pressed between guides 36a to 36c until the user decides to rotate lid 40e relative to base 20e.

FIGS. 11E and 11F are cross-sectional views of TMM device 10e highlighting certain interactions between base 20e, lid 40e and core sealer 60e. In particular, FIG. 11E illustrates both plugs 62 of core sealer 60e filling or plugging both loading apertures 520 and 52c of mating wall 42. Additionally, posts 48 of mating wall 42 of lid 40e are shown slidingly engaging peripheral posts 28 of bottom wall 22 of base 20e. Lid 40e and core sealer 60e are also illustrated fitting snugly against guide 36a (and do so likewise against guides 36b and 36c). FIG. 11F illustrates central plug 62 of core sealer 60e filling or plugging central loading aperture 52c of mating wall 42. FIG. 11F further illustrates cavity C residing between mating wall 42 and bottom wall 22 within which media grows as discussed in detail below.

Referring now to FIGS. 11G to 11J, an alternative TMM device 110e is illustrated, which may be made of any of the materials discussed herein. Alternative TMM device 110e includes many of the same components as TMM device 10e, which are numbered the same and include all structure, features and alternatives described above for those components. Specifically, TMM device 110e includes inner ring posts 26, outer ring posts 28, and guide posts 36a to 36c as illustrated in device 10e. For simplicity, inner ring posts 26, outer ring posts 28, and guide posts 36a to 36c are referenced herein collectively as a “ring formation.”

While sharing the ring formation with TMM device 10e and others, TMM device 110e is different from TMM device 10e and others because TMM device 110e provides modified structure and functionality. TMM device 110e places the ring assembly on a slide 25, which facilitates easier observation on microscopes equipped with a slide holder. TMM device 110e can also allow a user to observe multiple samples that are exposed to the same tissue culture media (growth factors, drugs, chemoattractants) if the separating wall 31 is removed as illustrated in FIG. 11J. Additionally, device 110e includes two ring formations, a slide 25, a housing 27, and a lid 35. The slide 25 provides a base for both the housing 27 and the ring formations. The housing includes a peripheral wall 29 and a divider wall 31, which separates the two ring formations. The peripheral wall 29 includes a lip 33, which engages lid 35. The lid 35 is used to cover the samples and keep them sterile.

FIG. 11H illustrates a cross-sectional view of TMM device 110e, which includes the two ring formations, slide 25, and housing 27. FIG. 11I illustrates an assembled cross-sectional view of TMM device 110e, which includes the two ring formations, the slide 25, the housing 27, the lid 35, the core sealer 60d, and the lid 40d. FIG. 11J illustrates an alternative implementation for TMM device 110e. Here, TMM device 110e is provided without a divider wall 31 located within housing 27.

Referring now to FIGS. 11K and 11L, an alternative TMM device 110d is illustrated. FIG. 11K illustrates the entire device 110d, while FIG. 11L is a cross sectional view of one of the plurality of the ring formations 37 located within its own well 41. Alternative TMM device 110d includes many of the same components as TMM device 10d, which are numbered the same and include all structure, features and alternatives described above for those components. Specifically, TMM device 110d includes a plurality of ring formations 37 located on a plate 39. Each ring formation 37 is located within its own well 41 (FIG. 11L).

Referring now to FIGS. 11M to 11P, an alternative TMM device 110a is illustrated, and which may be made of any of the materials discussed herein. Alternative TMM device 110a includes many of the same components as TMM device 10a, which are numbered the same and include all structure, features and alternatives described above for those components. TMM device 110a allows for full regulation of external factors and compression. FIG. 11M illustrates that TMM device 110a includes a slide 45, a slide base 47, a slide lid 49, and a slide core sealer 51. The slide 45 includes a ring formation, environmental sealers 50a and 50b, and regional dividers 46a and 46b. The slide lid 49 includes media loading apertures 520 and 52c, mounting holes 54, and loading holes 56. Slide core sealer 51 includes plugs 62 that plug loading apertures 52 of slide lid 49. Threaded bolts 32 extend through mounting holes 54 and thread into threaded mounting holes 30 formed or defined in slide base 47. Tank sealers 34 selectively plug loading holes 56 in the slide lid 49 when desired by the user.

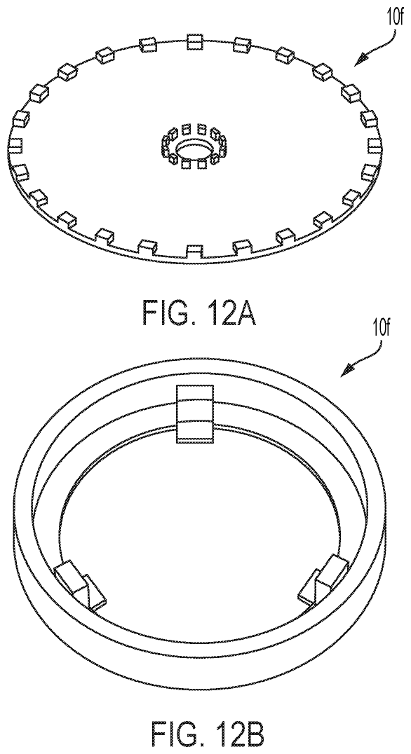

Referring now to FIGS. 12A and 12B, yet another alternative TMM device 10f is illustrated, which may be made of any of the materials discussed herein.

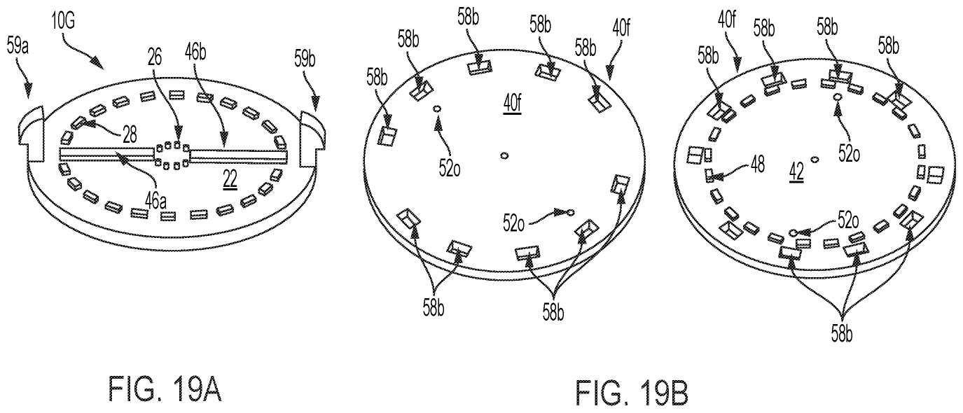

Referring now to FIGS. 19A and 19B, an alternative TMM device to 10d is illustrated, and which may be made of any of the materials discussed herein. Alternative TMM device 10g includes many of the same components as TMM device 10d, which are numbered the same and include all structure, features, and alternatives described above for those components. In contrast with the 10d, 10g does not include the guides 36a to 36c but has the guides 59a and 59b that allow snap-fitting of the 40f lid, through the cavities 58b which correspond to the “environmental openness” settings in a similar manner as do threaded holes 30 of TMM device 10b. Further, the regional dividers 46a and 46b are located at base 22 and extend from the core post (26) outwardly to the outer post (28), which provides a physical separation of the invasion zone, thereby creating more than one distinct invasion zone.

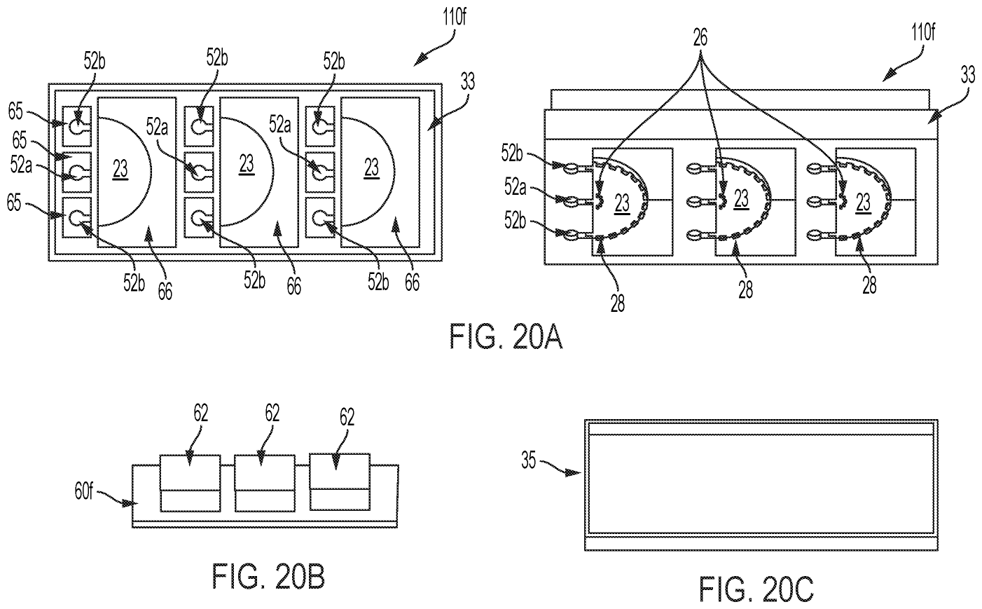

Referring now to FIGS. 20A to 20C, an alternative TMM device 110e is illustrated, and which may be made of any of the materials discussed herein. Alternative TMM device 110f includes many of the same components as TMM device 110d, which are numbered the same and include all structure, features and alternatives described above for those components. In contrast with the 110d, 110f does not include the bottom slide 25, as all features are attached to the peripheral wall 33, thus allowing the introduction of any see-through materials discussed herein to allow high resolution microscopy. Additionally, the post 26 and 28 are attached on the top wall 23 and form two concentric semi-circles and are located in individual wells 66. Further, the loading guides for the ECM mimic 52b and core loader 52a are located in individual wells 65 and are connected either with the invasion zones or the core sample are respectively. Each well set of loading guides is sealed with the use of the lid 60f, by forming a tight seal between the wells 65 and plugs 62. The lid 35 is used to cover the samples and keep them sterile.

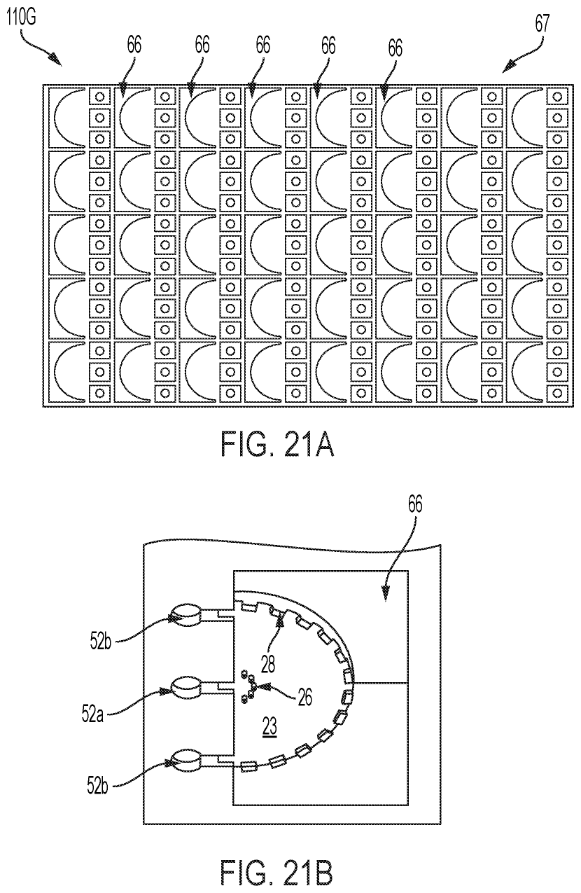

Referring now to FIGS. 21A and 21B, an alternative TMM device 110d is illustrated and a multiwell embodiment of 110f, and which may be made of any of the materials discussed herein. Alternative TMM device 110g includes many of the same components as TMM device 110f, which are numbered the same and include all structure, features and alternatives described above for those components.

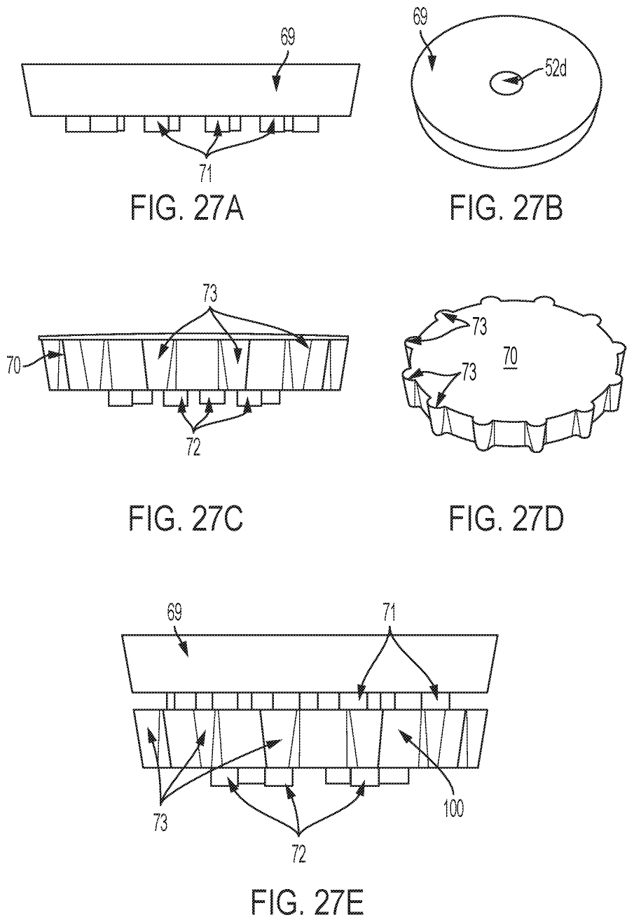

FIGS. 27A to 27E, illustrate the TMM device 10H, which may be made of any of the materials discussed herein, as a two-tiered embodiment of the TMM devices 10e and 10f. TMM device 10H includes a base (69) as its top layer (FIGS. 27A and 27B) that has cylindrical posts which form a ring and are extended to mate with the bottom base (70) to form the invasion zone of the TMM device 10H. Also, there is an ECM mimic loading opening (52d), which is located at the center of the base 69, to allow the introduction of any ECM mimic and any cell type within the invasion zone of the TMM device 10H after its setup. Moreover, the top layer (69) acts as a sealer for the invasion zone of the TMM device 10H but allows both nutrient and oxygen intake via the ECM mimic loading opening (52d). Variable intake can be achieved through variation of the diameter of this opening and intravasation mimicked by covering the intake opening with a porous polyester membrane and the introduction of an endothelial cell monolayer over the membrane. Additionally, the bottom base (70) has cylindrical posts (72), which form a ring and define the core area of the TMM device 10H, similar to the pre-described core posts 26. Also, the bottom base (70) has small extrusions (73) to its periphery to allow diffusion of nutrients and oxygen to the core area.

Devices 10a to 10h, 110a, 110d, 110e, 110f, and 110g may be sterilized via any method known to a person of skill in the art. Additionally, at least TMM devices 10a to 10g may be used with petri dishes and/or multi-well formats, including, but not limited to two, four, six, twelve, twenty-four, forty-six, and/or ninety-six well plates. Any one or more or all of TMM devices 10a to 10g may also be scaled up or down to size as needed in order to be inserted into any petri dish, multi-well plate format (for example, as shown in FIGS. 11M to 11P and 11G to 11I), and/or a microscope slide holder format (for example, as shown in FIGS. 11M to 11P and 11G to 11I). Further additionally or alternatively, the TMM device features can be directly introduced into any petri dish, multi-well plate format (for example, as shown in FIGS. 11M to 11P and 11G to 11I), and/or microscope slide holder format (for example, as shown in FIGS. 11M to 11P). A suitable lid or covering is used to set the desirable intake of media and/or oxygen.

Additionally, the features of any of the TMM devices 10a to 10g can be produced directly in wells of a multi-well plate (for example, as shown in FIGS. 11M to 11P and 11G to 11I) or on a microscope slide format (for example, as shown in FIGS. 11M to 11P). Alternatively, the base posts (26 and 28) can be transferred to a base (23) that mates with a section of the peripheral wall to produce a multi-well format (for example, as shown in FIGS. 21A and 21B) or a microscope slide format (for example, as shown in FIG. 20A), which has no bottom to allow post production introduction of a transparent base, which can be produced by any of the materials discussed herein. Finally the major features can also be established in a 96 well plate format that allows high throughput screenings through the use of a two tiered device as shown in FIGS. 27A to 27E.

### TMM Methods

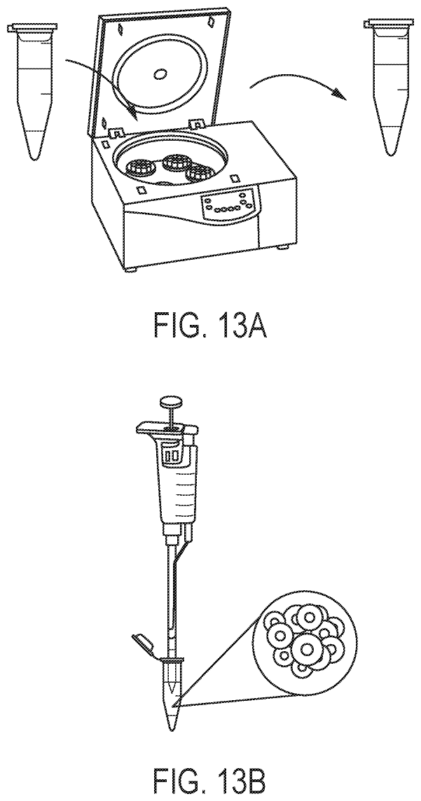

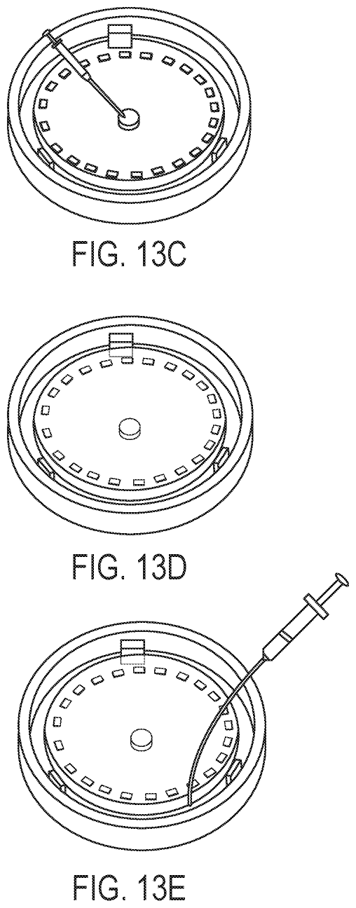

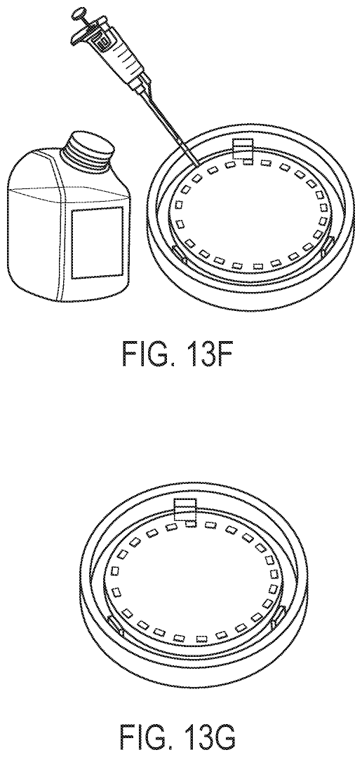

Any of the TMM devices 10a to 10h, 110a, and 110d to 110g may be used to mimic the microenvironment of a tumor and investigate cell invasion, cell migration, and/or cell responses to various environmental factors or other cells. Additionally, any of the TMM devices 10a to 10h, 110a, and 110d to 110g may be used for the isolation of cancer cell subpopulations of increasing aggressiveness. Cells of interest can be deposited into the sample core area via the opening 52c of any of the TMM devices 10a to 10g, 110a, 110d, 110e, 110f, and 110g as shown in FIGS. 13A to 13E, which illustrate one embodiment of a method of the present disclosure for preparing a microtumor assay protocol. In FIG. 13A, cells of interest are subjected to centrifugation and the supernatant is removed. In FIG. 13B, a pellet of cells is resuspended in order to achieve a final concentration between 5,000 to 150,000 cells/μl or more preferably, about 40,000 to about 80,000 cells/μl, for example, within a selected hydrogel polymer. In FIG. 13C, the resuspended cells are delivered or added to a sample core of any of devices 10a to 10h, 110a, 110d, 110e, 110f, and 110g.

In FIG. 13D, a round glass coverslip or the appropriate lid (for any TMM devices 10a to 10g, 110a, 110d, 110e, 110f, and 110g) may be placed so as to cover the delivered cells. For devices 10a to 10g, lids 40a to 40f are placed onto their respective base 20a to 20e, wherein the user rotates lid 40a to 40f to a desired “environmental openness” setting for the media and/or oxygen relative to the respective base 20a to 20e in a manner illustrated above. Core sealers 60a to 60e may then be placed onto respective lids 40a to 40e to plug their openings 52.

In FIG. 13E, a selected extracellular matrix (“ECM”) mimic is introduced to the invasion zone area of device 10a to 10h, 110a, 110d, 110e, 110f, and 110g via side openings (FIG. 13E). In FIG. 13F, device 10a to 10h, 110a, 110d, 110e, 110f, and 110g is placed again in the incubator for polymerization if necessary, for example when using some hydrogels as the selected ECM. Cell culture media is added to the media tank of device 10a to 10h, 110a, 110d, 110e, 110f, and 110g. In FIG. 13G, device 10a to 10h, 110a, 110d, 110e, 110f, and 110g is ready for use and can be observed in the incubator for further analysis of cell 3D migration and/or invasion.

As the cells within the sample core of device 10a to 10h, 110a, 110d, 110e, 110f, and 110g consume nutrients and oxygen, the immediate microenvironment within the sample core can mimic a necrotic core or a hypoxic area of a tumor. Generally, the invasion zone surrounding the sample core provides fewer environmental challenges compared to the sample core area, in part due to a larger surface area. Thus, the invasion zone area can provide a more desirable microenvironment for the cells within the sample core. Environmental factors may include, but are not limited to oxygen, pressure, cell nutrients, other cells, chemoattractants, and/or growth factors. Cells within the sample core may migrate or invade the invasion zone area. Additionally, or alternatively, one or more of the environmental factors within the invasion zone may be less desirable, rather than more desirable, in comparison to the sample core.

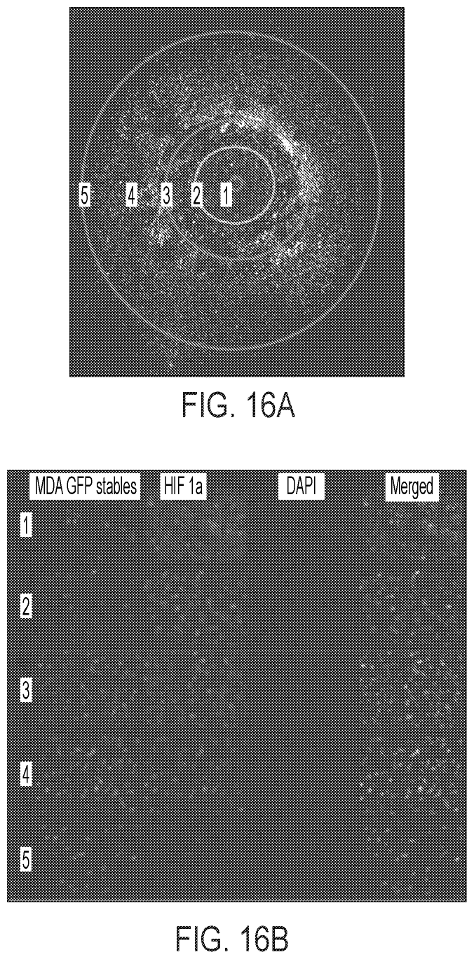

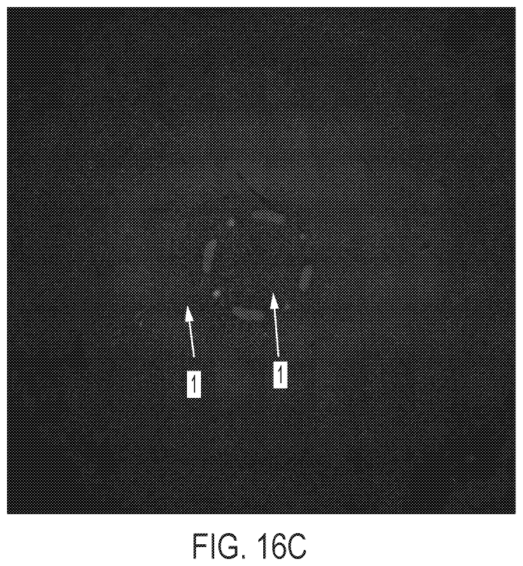

FIGS. 16, 22, 23, 17, and 18 illustrate that the microenvironment within any of the TMM devices 10a to 10h, 110a, 110d, 110e, 110f, and 110g have the ability to replicate the in vivo tumor phenotype and microenvironment in vitro, as they in any of the TMM devices are characterized by the establishment of oxygen, pH, and nutrient gradients, which induces the generation of a necrotic core within a 3D environment, respectively.

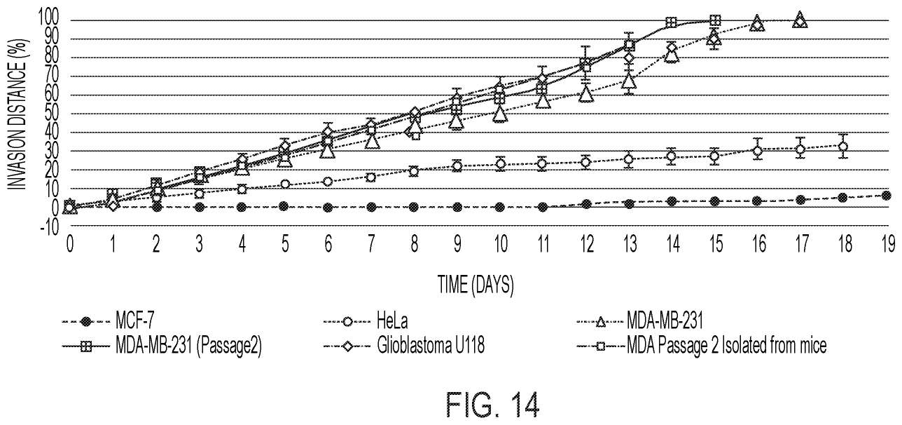

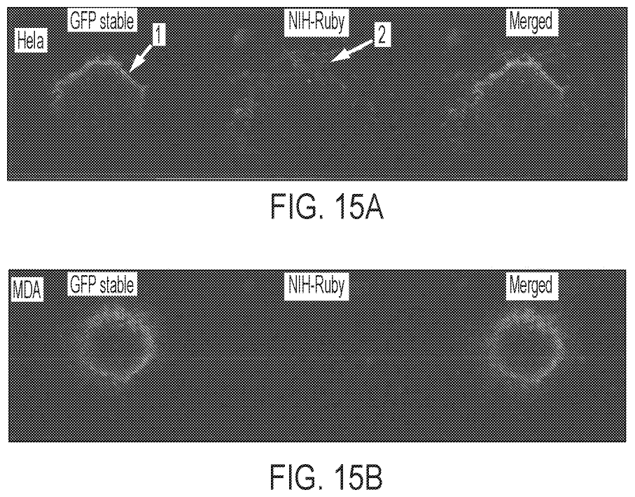

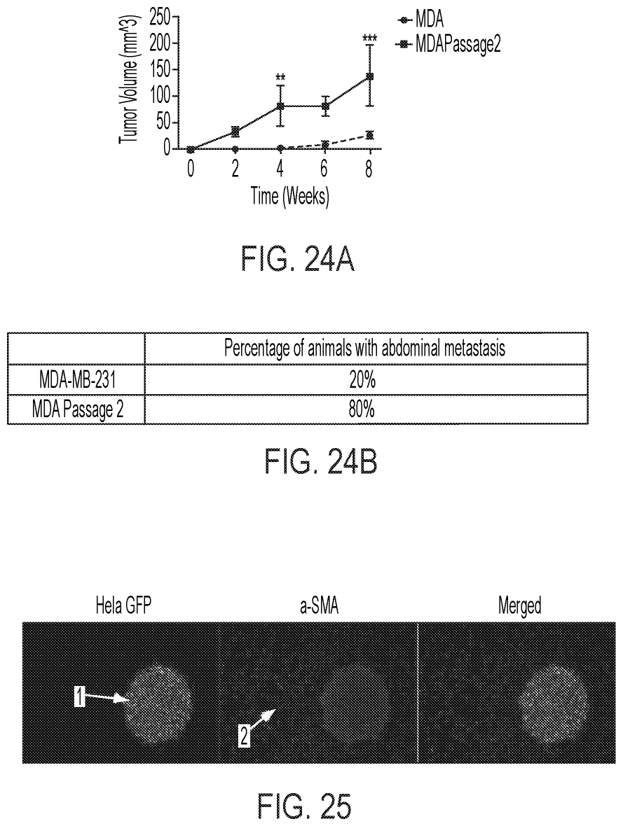

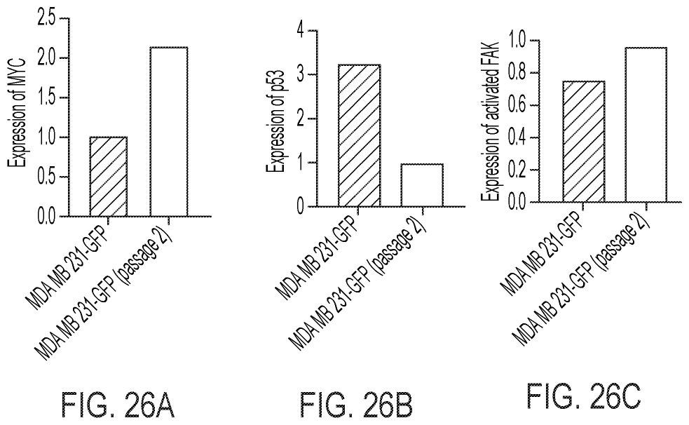

Additionally, the accurate replication of the in vivo tumor microenvironment, described above, enables the isolation of cancer cell subpopulations that exhibit higher metastatic potentials. FIG. 14 shows the in vitro comparison of metastatic potentials within the TMM device 10a for the parental MDA-MB-231 cell line and MDA TMM 2 cell line, which are MDA-MB-231 cells that have been introduced to the 10a TMM device core and were allowed to traverse and exit the invasion zones of the device and subsequently isolated from the device periphery, the process was repeated. FIG. 14 illustrates that MDA TMM 2 cells have higher in vitro metastatic potential than their parental cell line, as they are able to traverse and exit the invasion zones of the TMM device significantly faster. Also, FIGS. 24A and 24B illustrate that MDA TMM 2 cells display far more aggressive characteristics than the parental lines when used in xenograft experiments in which they displayed faster tumor establishment, formation of larger primary tumors, and higher in vivo metastatic potential. Moreover, FIGS. 26A to 26C show that MDA TMM 2 cells display elevated expression of proteins shown to promote tumor aggressiveness and progression, such as the c-Myc oncoprotein and phosphorylated FAK (pFAK), and reduced expression of the tumor suppressor p53, which can prevent cancer cells from acquiring additional mutations, by inducing cell death, and its downregulation is associated with worse prognosis in cancer patients.

Any of the TMM devices 10a to 10h, 110a, and 110d to 110g can be used for the isolation of cancer cell subpopulations from existing and already characterized cancer cell lines. This is a unique characteristic of the disclosed device and methods none of the in vitro assays in use today have been demonstrated to allow the isolation of a heterogeneous subpopulation of cancer cells that exhibit in vivo validated increased aggressiveness. Also, currently, isolation of most metastatic subpopulations or variants is carried out in animal models by introducing the cancer cells in the animals and isolating the metastatic foci, which is an extremely time-consuming, labor-intensive, and expensive process. Therefore, the disclosed device and methods could be used as an alternative method to the animal models for the isolation of more aggressive cell subpopulations. The isolation of such subpopulations with any of the TMM devices can enable the direct and accurate determination of gene expression changes, epigenetic modifications and metabolic changes responsible for the increased aggression. Such studies are complicated when carried out in vivo because the establishment of metastatic foci at a distal organ can significantly alter the expression profile of these cells due to the dramatically different conditions at these sites, compared to the original tumor location. This includes different growth factors, interacting cell types and biomechanical differences such as tissue stiffness and mechanical stimuli. These gene expression changes which are a consequence of cancer cell growth at a distal site complicate the identification of gene expression changes as they can mask gene expression, epigenetic, and metabolic changes that lead to increased aggressiveness. However, these limitations can be eliminated when cancer cells subpopulations are isolated using any of the TMM devices because they only expose the cells of interest to environmental stressors and not to variable mechanical stimuli or different tissue specific signaling molecules.

Therefore, comparison of isolated cancer cell subpopulations using the disclosed devices, with their parental cell lines and to each other can be used to understand and map genomic and transcriptomic alterations in cancer cells and is of crucial importance since it will not only enhance our understanding of tumor cell progression, but can also uncover, diagnostic markers as well as prognostic markers enabling early diagnosis and facilitating efficient treatment. In addition, cell subpopulations derived using the disclosed devices can be used for high-throughput studies for the characterisation of antimetastatic agents. Also, as any of the TMM devices 10a to 10h, 110a, and 110d to 110g can recapitulate the in vivo tumor microenvironment and can differentiate between metastatic and non-metastatic cells, they can be used to accurately evaluate novel anti-cancer medications in a relevant environment, used as a fast and accurate diagnostic tool, and as a tool for testing different anti-cancer medications for personalized treatments.

Furthermore, any of the TMM devices 10a to 10h, 110a, 110d, 110e, 110f, and 110g may be used to co-culture at least one type of cell and investigate the cell interactions and/or the affect the additional cells may or may not have on the migration or invasion of tumor cells from the sample core to the invasion zone. In one embodiment, fibroblasts may be added to the invasion zone. In another embodiment, endothelial or epithelial cells may be added to the periphery of the invasion zone in order to study tumor cell intravasation. Furthermore, in an additional embodiment, more than one ECM material and/or additional cell type can be added to distinct regions of the invasion zone.

The invasion zone may also contain any material that can resemble or mimic an extracellular matrix (“ECM”). An ECM mimic may be deposited into the entire area of the invasion zone or distinct areas within the invasion zone of any of the TMM devices 10a to 10h, 110a, 110d, 110e, 110f, and 110g. The ECM mimic can be introduced into the TMM device during the initial setup of the device as shown in FIG. 13E or the ECM mimic can be deposited via one of the openings 52. In one embodiment, Matrigel® matrix can be added to the invasion zone of any of the TMM devices 10a to 10h, 110a, 110d, 110e, 110f, and 110g. In another embodiment, collagen can be added to the invasion zone of any of the TMM devices 10a to 10g. The ECM mimic can include any biocompatible hydrogel or material at any acceptable concentration, known to a person of skill in the art.

Solutions or media can be added to the media reservoir or media tank of any of the TMM devices 10a to 10h, 110a, 110d, 110e, 110f, and 110g via the loading holes 56. Additionally or alternatively, any of the TMM devices 10a to 10g, 110a, 110d, 110e, 110f, and 110g, which can be inserted into any petri dish, multi-well plate format (for example, as shown in FIGS. 11M to 11P, 11G to 11I, and 21A and 21B), and/or microscope slide holder format (for example, as shown in FIGS. 11M to 11P, and 20A to 20C), can also be submerged in a media that is added to the well, dish, or holder itself. Any solution or media known to a person of ordinary skill in the art may be used. Various volumes of media and/or solutions known to a person of ordinary skill in the art may be added to the media reservoir or media tank. The solution or media may be replaced at any time by removing the solution or media via loading holes 56 then adding the replacement or new media via the loading holes 56.

In one embodiment, growth factors can be added to the media located in the media tank via loading holes 56 of any of the TMM devices 10a to 10g, 110a, 110d, 110e, 110f, and 110g. The concentration of growth factors in the media may be any concentration known to a person of ordinary skill in the art.

In one embodiment, regional dividers 46a and 46b extend outwardly from the mating wall 42 and provide a physical separation of the invasion zone, thereby creating more than one distinct invasion zone in any one of the TMM devices 10a to 10g. The distinct invasion zones of TMM device 10a and/or TMM device 10b, for example, may contain different ECM material and/or other cell types within each invasion zone. Thereby, by analyzing the migration or invasion pattern or distance of the cells of interest (from the sample zone into the invasions zones), the effects of one invasion zone compared to the other invasion zone can be analyzed.

In another embodiment, the cells within TMM devices 10a to 10h, 110a, 110d, 110e, 110f, and 110g can be visualized using microscopy techniques. TMM devices 10a to 10h, 110a, 110d, 110e, 110f, and 110g can be scaled up or down to a size necessary to accommodate a desired microscope slide (for example, as shown in FIGS. 11M to 11P and 11G to 11I) can be placed on a microscope slide or other suitable surface and the cells can be visualized live and/or visualized following staining, fixing, or labeling using any process or procedure known to a person of skill in the art. Also, another embodiment of the TMM devices can be produced as a slide or a multi-well without a base (FIGS. 20A to 20C, 21A, and 21B, respectively) to allow the introduction of a glass bottom for high resolution microscopy. Any suitable microscopy technique that is known to a person of ordinary skill in the art may be used.

In one embodiment, a tumor cell line of interest is added to the sample core of any of the TMM devices 10a to 10h, 110a, 110d, 110e, 110f, and 110g, at a concentration of about 5,000 cells/μl to about 150,000 cells/μl or more preferably, about 40,000 to about 80,000 cells/μl. The tumor cells are allowed to grow, proliferate, and invade until they exit the invasion zone of the TMM device. This process can take between 10 days to 90 days depending on the type and concentration of the ECM mimic introduced to the invasion zone of the TMM device, and the metastatic potential of the cells introduced in the TMM device core. After migrating tumor cells exit the invasion zone, they can be easily isolated by trypsinization. The cell isolation process (also referred to as cell passage through the invasion zones of the device) may be carried out once, twice, three times, four times, five times, or six times, in order to generate progressively more metastatic subpopulations. The method of passaging the tumor cells is generally known to a person of ordinary skill in the art.

In another embodiment, a tumor cell line of interest is added to the sample core of any of the TMM devices 10a to 10h, 110a, 110d, 110e, 110f, and 110g. and the ability of the tumor cell line of interest to migrate or invade the invasion zone can be assessed over time. This method may distinguish between metastatic and non-metastatic cell lines.

In another embodiment, the effects of various environmental factors present within the invasion zone of any of the TMM devices 10a to 10h, 110a, 110d, 110e, 110f, and 110g on the ability of a tumor cell of interest to migrate or invade the surrounding area is analyzed.

In another embodiment, anti-cancer drugs or other test drugs or compounds can be evaluated by analyzing their effects on the ability of tumor cells to migrate or enter the invasion zone of any of the TMM devices 10a to 10h, 110a, 110d, 110e, 110f, and 110g in the presence of the test drug or compound. The concentration of test drugs or compounds may include any concentration known to a person of skill in the art. The concentration range of test drugs or compounds provided to the tumor cells within any of the TMM devices 10a to 10h, 110a, 110d, 110e, 110f, and 110g may include an approximate concentration of test drug or compound that is effective at slowing migration of the tumor cells.

From the core of any of the TMM devices 10a to 10h, 110a, 110d, 110e, 110f, and 110g in the present disclosure, stable radial gradients of hypoxia, acidosis, and nutrients are established. Thus, in another embodiment, cells can be uniformly introduced throughout the invasion zone and core of any of the TMM devices 10a to 10g, 110a, 110d, 110e, 110f, and 110g in the present disclosure, at a concentration of about 5,000 cells/μl to about 150,000 cells/μl or more preferably, about 40,000 to about 80,000 cells/μl, to study their differential metabolic activity under a spectrum of metabolic conditions.

### EXAMPLES

### Example 1

Using TMM device 10c, for example, the migration distance of six different cell lines was compared to evaluate the TMM device's abilities to distinguish between cell lines with different metastatic potential. The following cell lines were deposited into individual TMM devices, containing 2% collagen as the ECM, (1) non-metastatic MCF-7 cells, (2) invasive but not metastatic (Malignant tumor) HeLa cells, (3) metastatic MDA-MB-231 cells, and (4) metastatic Glioblastoma U118 cells, (5) highly metastatic subpopulation of MDA-MB-231 cells (MDA-TMM 2) isolated from the TMM device, and (6) MDA-TMM 2 cells isolated from a mouse primary tumor.

The migration distance of the cells in each device was measured daily until the cells reached the outer perimeter of the invasion zones, except for the MCF-7 and Hela cells whose monitoring ended after 19 and 18 days, respectively, as illustrated in FIG. 14. Data points in FIG. 14 represent the mean (+SD) of 30 measurements (n=30) for each corresponding point in time. The migration distance (μm) in the illustrated embodiment was measured from the outer edge of the sample core to the farthest cell from the edge of the sample core. Thirty distance measurements were taken for each device and for each timepoint. The measurements were averaged, and the standard deviation was calculated. FIG. 14 shows that MCF-7 cells, which are non-metastatic, completely fail to invade and display extensive cell death, via both necrosis and apoptosis. HeLa cells, which have been shown to be invasive but not metastatic (Malignant tumor), are able to invade very slowly and to a limited depth. On the other hand, MDA-MB-231 and U118, which both are highly metastatic cell lines, display clear and sustained invasion. Additionally, MDA TMM 2, which are MDA-MB-231 cells that have been introduced to the 10a TMM device core and were allowed to traverse and exit the invasion zones of the device twice, showed to have higher metastatic potential than their parental cell line. Moreover, MDA TMM 2 cells were introduced in nude mouse models and after 9 weeks the primary tumors were surgically removed, which allowed re-isolation of MDA TMM 2 cells. Then the re-isolated cells were re-introduced to the core of the TMM device and showed identical invasion abilities with the MDA TMM 2 cells that were not introduced within the animal models. Thus cells that have traversed the device are stably modified compared to the parental cell line.

The data illustrate that the disclosed device and in vitro methods are able to distinguish between benign, malignant, and metastatic cells. Also, this experiment shows that the disclosed device and in vitro methods allow the introduction and testing of the invasive capacity of any human tumor cell line or primary tumor cell meshes from biopsies in a controlled and reproducible manner.

### Example 2

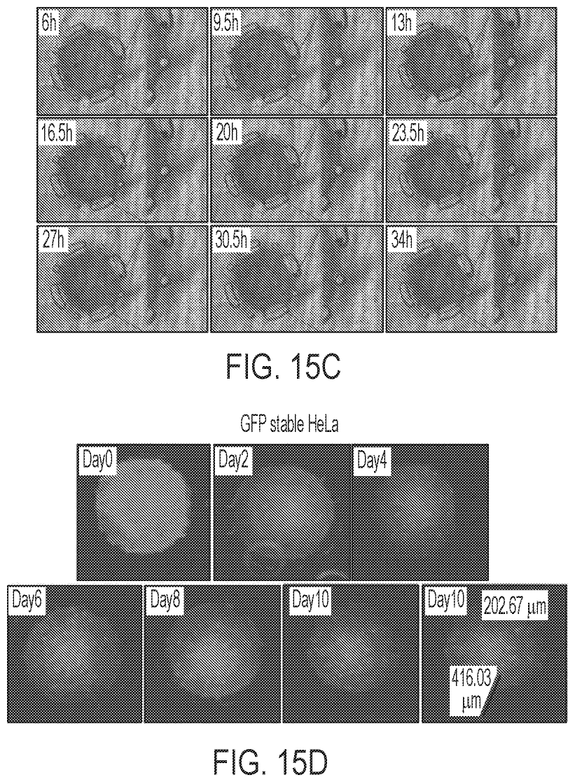

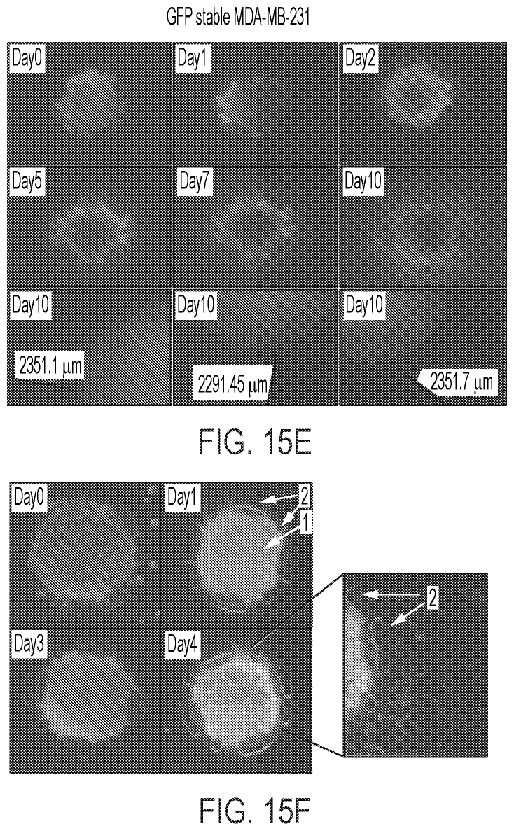

Using the TMM device 10e, the migration distance of Hela or MDA-MB-231 cells were co-cultured with NIH/3T3 mouse embryonic fibroblasts. The Hela MDA-MB-231 cells, expressing GFP, were visualized using fluorescence after three (3) days in culture. The NIH/3T3 mouse embryonic fibroblasts were stained with Ruby within the invasion zone. FIGS. 15A to 15F show the migration of HeLa MDA-MB-231 cells, respectively, from within the sample core of the TMM device, labeled with GFP, and NIH/3T3 mouse embryonic fibroblasts stained with Ruby within the invasion zone. In particular, FIG. 15A shows Hela cells (1) within the sample core of the TMM device, labeled with GFP, and NIH/3T3 mouse embryonic fibroblasts (2) stained with Ruby within the invasion zone. FIGS. 15B and 15F show the MDA-MB-231 cells (1) within the sample core of the TMM device labeled with GFP, and NIH/3T3 mouse embryonic fibroblasts (2) stained with Ruby within the invasion zone. Specifically, FIG. 15B illustrated that using the 10e the NIH/3T3 mouse embryonic fibroblasts can be introduced to half of the invasion zones in order to evaluate with the same samples how the cells behave in the presence or absence of fibroblast within the invasion zones of the device. Further, Hela cells, expressing GFP were introduced to the core of TMM device 10e and were co-cultured with normal NIH/3T3 mouse embryonic fibroblasts, which were introduced within the invasion zones of the device, for a period of 8 days. FIG. 25 illustrate the conversion of normal NIH/3T3 to cancer associated fibroblasts (1) (CAFs) after 8 days, via immunofluorescence staining with a mouse monoclonal anti-Smooth-muscle actin antibody, which is the most widely accepted marker for CAFs, and a secondary anti-mouse 647 (2) antibody. Therefore, overall, these experiments show that the disclosed device and methods allow for the introduction of multiple cell types within the invasion zones and co-culture them with cancer cells in order to study their role in metastasis.

### Example 3

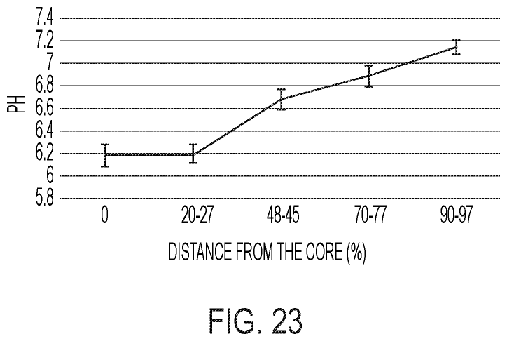

FIGS. 23 and 16C show an embodiment, in which MDA-MB-231 cells (1) were introduced uniformly within the TMM device's invasion zones in order to evaluate the microenvironment and gradients generated by the disclosed device and methods. Specifically, FIG. 23 shows the pH gradient that is formed within the device after 24 hours using the 5-(and -6)-Carboxy SNARF™-1, Acetoxymethyl Ester, Acetate (Invitrogen). This illustrates that the core of the device and the initial areas of the invasion zones are highly acidic with a pH of about 6.2, which is a characteristic of almost all solid tumors in vivo. Also, FIG. 23 shows that pH rises linearly within the invasion zone until it reaches the end of the invasion zones with a pH of about 7.2, which is close to the physiological conditions in vivo. Furthermore, FIG. 16C shows the oxygen gradient generated within the device after 24 hours using Image-iT™ Red Hypoxia Reagent (Invitrogen). FIG. 16C illustrates a strong red signal that derives from the MDA-MB-231 cells at the core and the beginning of the invasion zones, which indicates a hypoxic environment, whereas the red signal is reduced and then becomes lost from the end of the invasion zones.

FIGS. 16A and 16B show an embodiment, evaluation of a hypoxia gradient generated after twenty days of culture in any of 10a to 10g, 110a, 110d, 110e, 110f, and 110g devices of the present disclosure, in order to illustrate that the gradients within the TMM device are maintained over time. Metastatic MDA-MB-231 cells expressing GFP were added to the sample core of the device, and twenty days later, the cells were analyzed for the expression of HIF-1a, a marker for indication of hypoxia, via immunofluorescence. FIG. 16A shows the maximum intensity projection of migrating MDA-MB-231 cells within the device after the twenty days duration in the culture. FIG. 16B shows immunofluorescence images (rows 1-5) of MDA-MB-231 cells, which have been taken from the sample core, and four concentric areas of the invasion zone. The numbered rings shown in FIG. 16A correspond to the numbered rows of immunofluorescence images in FIG. 16B. This experiment illustrates a similar oxygen gradient with the Image-iT™ Red Hypoxia Reagent, thus suggesting that the de novo generated gradients with the disclosed device are maintained over long time periods.

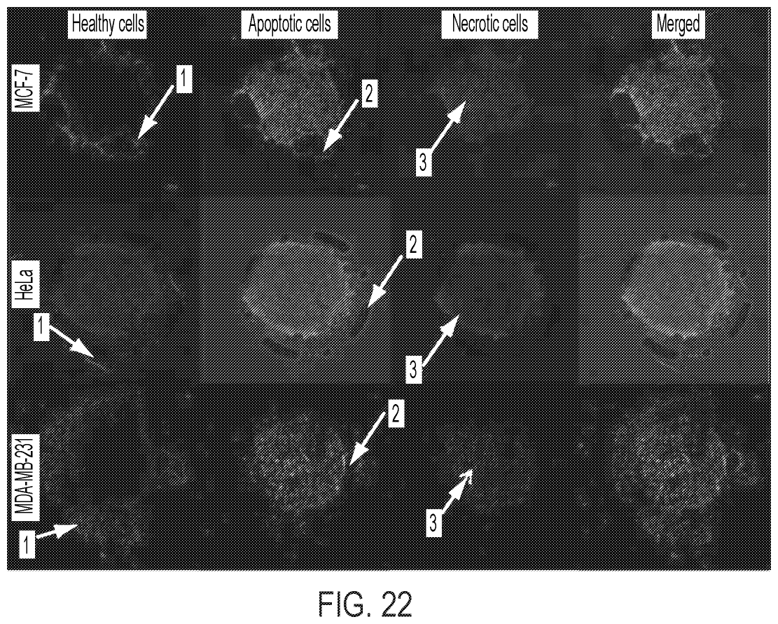

Using the TMM device 10c, for example, the formation of a necrotic core was evaluated using three cell lines. The following cell lines were deposited into individual TMM devices, containing 2% collagen as the ECM, (1) non-metastatic MCF-7 cells, (2) invasive but not metastatic (Malignant tumor) HeLa cells, (3) metastatic MDA-MB-231 cells. Specifically, two days after the cell introduction within the core of the TMM device the necrosis/apoptosis assay kit (Abcam, ab 176749) was used to evaluate live the formation of central necrosis. FIG. 22 shows that all three cell lines form extensive necrosis (3) and apoptosis (2) within the central region of the core sample, which is a common characteristic of almost all solid tumors. Additionally, a secondary ring is formed by the MCF-7 cells with healthy cells (1), which is another characteristic of solid tumors in vivo, whereas the healthy cells of the metastatic MDA-MB-231 cells can be observed also within the invasion zones.

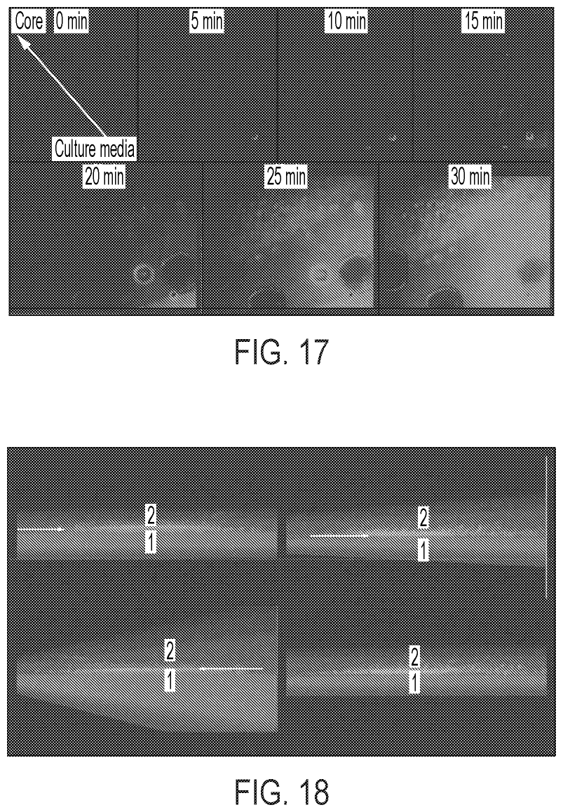

FIG. 17 shows another example of time-lapse imaging of the de-novo gradient of cell culture media growth towards the center of TMM device 10a to 10g, 110a, 110d, 110e, 110f, and 110g over a thirty-minute period. The cell culture media is visible due to staining with fluorescein isothiocyanate (“FITC”).

FIG. 18 shows in a further example, a depth-coded reconstruction of cell 3D migration from the sample core into the invasion zone of a TMM device 10a to 10g, 110a, 110d, 110e, 110f, and 110g. Layer 1 represents the lower boundary of the TMM device 10a to 10g, 110a, 110d, 110e, 110f, and 110g, while Layer 2 represents the upper boundary of device 10a to 10g, 110a, 110d, 110e, 110f, and 110g. The arrows indicate the direction of the migrating cancer cells through the invasion zone and the ECM mimic. Thus, FIG. 18 shows that the disclosed device and in vitro methods generate a 3D environment for the migrating cancer cells.

Therefore, these experiments illustrate that the disclosed device and methods can recapitulate and maintain the in vivo tumor microenvironment in vitro.

### Example 4

GFP expressing MDA-MB-231 metastatic cancer cells were deposited to the core of the TMM device 10e, containing 2% collagen as the ECM in both the core and the invasion zone, and the device was submerged into cell culturing media. Then the device was placed into an incubator until a small subset of cells traversed and exited the invasion zone. These cells exhibiting faster invasion and exit from the device were isolated and termed MDA TMM 1 cells. Next, the MDA TMM 1 cells were reintroduced to the core of the TMM device 10e and the procedure was repeated, and the MDA TMM 2 were isolated and compared with the parental cell line.

Specifically, FIG. 14 shows that the MDA TMM 2 cells have higher invasive potential compared to their parental cell line in vitro. Additionally, FIGS. 26A to 26C illustrate that the MDA TMM 2 cell line displays elevated expression of both c-Myc oncoprotein and phosphorylated FAK (pFAK), which have been shown to promote tumor aggressiveness and metastasis. Furthermore, the MDA TMMe 2 cell line shows decreased expression of the tumor suppressor protein p53, which is a protein that can prevent cancer cells from acquiring additional mutations, and its downregulation is associated with worse prognosis in cancer patients. Further, FIG. 24A and FIG. 24B illustrate that animals injected with the MDA TMM 2 cell line form tumors faster and that are significantly larger compared to the parental line. Also, FIG. 24B shows that 80% of the animals injected with the MDA TMM 2 cell line formed abdominal metastases, whereas only 20% of the animals injected with the MDA-MB-231 cell line formed abdominal metastases. Overall, these experiments illustrate that the disclosed device and methods apply selective pressures to the core sample, which allow the isolation of cancer cell subpopulations that exhibit higher aggressiveness and metastatic potential both in vitro and in vivo, and also have a differential protein expression compared to the initial sample. The isolation of cancer cell subpopulations is a procedure typically carried out in vivo because currently, it is difficult to do with other in vitro devices, as typically are used for single-cell isolations or for isolating cells based on cell surface markers, which are already known.

It should be understood that various changes and modifications to the presently preferred embodiments described herein will be apparent to those skilled in the art. For example, while lid posts 48 are illustrated as fitting inside base posts 28, base posts may instead have a smaller diameter and fit inside lid posts 48. Such changes and modifications can be made without departing from the spirit and scope of the present subject matter and without diminishing its intended advantages. In another example, while indentations 58 are illustrated as being provided in lids 40b and 40d, the indentations 58 may alternatively be provided in corresponding bases 20b and 20d, while the snap-fittingly mating projections 38 are provided in lids 40b and 40d. It is therefore intended that such changes and modifications be covered by the appended claims.

The invention is claimed as follows:

1. A tumor microenvironment mimicking (“TMM”) device configured to restrict both nutrient and oxygen intakes the TMM device comprising:
a base including a bottom wall and a plurality of base posts extending from the bottom wall,
the base posts separated from each other to form spaces between the base posts; and
a lid sized to mate with the base, the lid including a mating wall and a plurality of lid posts extending from the mating wall, the lid posts separated from each other to form spaces between the base posts, the lid posts positioned when the lid is mated with the base so as to be able to variably cover the spaces between the base posts based on a rotational position of the lid relative to the base.

2. The TMM device of claim 1, wherein the base posts are extended downward from a base, wherein the base is mounted onto a peripheral wall and a bottom wall, and wherein the bottom wall is mated to the peripheral wall and the base posts.

3. The TMM device of claim 1, wherein the base posts form a ring of base posts and the lid posts form a ring of lid posts, and wherein the ring of lid posts is slightly larger or smaller than the ring of base posts.

4. The TMM device of claim 3, wherein the rings are co-centric rings, and wherein the inner or outer diameter of the ring of lid posts is sized to slidingly engage the outer or inner diameter, respectively, of the ring of base posts.

5. The TMM device of claim 1, wherein the base posts and the lid posts have at least substantially the same height.

6. The TMM device of claim 1, wherein the base posts are outer base posts, and wherein the base further includes inner core posts extending from the bottom wall, the inner core posts separated from each other to form spaces between the core posts.

7. The TMM device of claim 6, wherein the inner core posts form a ring.

8. The TMM device of claim 1, wherein the lid and the base are configured to releasably fix the rotational position of the lid relative to the base.

9. The TMM device of claim 7, wherein the lid is configured to be fastened to the base to releasably fix the rotational position of the lid relative to the base.

10. The TMM device of claim 9, wherein the mating wall extends from a sealing flange of the lid and a peripheral wall extends from the bottom wall of the base, and wherein the sealing flange is configured to be fastened to the peripheral wall to releasably fix the rotational position of the lid relative to the base.

11. The TMM device of claim 9, wherein the fastening of the lid to the base includes a plurality threaded apertures, each aperture corresponding to a different environmental openness setting.

12. The TMM device of claim 8, wherein one of the lid or base includes a projection that snap-fittingly mates with one of a plurality of indentations provided by the other of the lid or the base to fix the rotational position of the lid relative to the base.

13. The TMM device of claim 12, wherein each of the indentations corresponds to a different environmental openness setting.

14. The TMM device of claim 1, wherein the lid defines at least one media loading aperture for injecting media into a cavity formed between the mating wall of the lid and the bottom wall of the base.

15. The TMM device of claim 14, further comprising a core sealer sized to be placed on the lid, the core sealer including at least one plug that plugs the at least one media loading aperture.

16. The TMM device of claim 1, wherein the mating wall extends from a sealing flange of the lid, and wherein the sealing flange defines at least one loading hole for receiving media and/or oxygenradially outside of the spaces and the base posts when the lid is mated with the base.

17. The TMM device of claim 16, further comprising at least one media tank sealer for plugging the at least one loading hole.

18. The TMM device of claim 16, further comprising at least one divider extending radially from the mating wall and perpendicularly from the sealing flange, the at least one divider blocking a space between the mating wall of the lid and a peripheral wall of the base when the lid is mated to the base.

19. The TMM device of claim 1, wherein the lid includes at least one divider extending from the mating wall, the at least one divider enabling different cells to be placed on the bottom wall of the base, the at least one divider enabling the different cells to be separated.

20. The TMM device of claim 19, wherein the at least one divider includes two aligned dividers that at least substantially bifurcate the mating wall.

21. The TMM device of claim 1, wherein the lid includes at least one divider extending from the mating wall, the at least one divider enabling different cells to be placed on the bottom wall of the base, the at least one divider enabling the different cells to be separated.

22. The TMM device of claim 19, wherein the at least one divider includes two aligned dividers that at least substantially bifurcate the mating wall.

23. A method of assessing tumor cell migration, the method comprising:
providing tumor cells to a sample core of the TMM device of claim 1;
growing the tumor cells; and
measuring distance, speed, and number of the tumor cells that migrate away from the sample core and into an invasion zone.

24. A method of isolating a heterogeneous cancer cell subpopulation, the method comprising:
providing tumor cells to a sample core of the TMM device of claim 1;
enabling the tumor cells to exit the sample core and enter an invasion zone of the TMM device of claim 1; and
isolating the tumor cells from the invasion zone.
